(19) United States
(12) Patent Application Publication
(10) Pub. No.: US 2023/0087351 A1
(43) Pub. Date: Mar. 23, 2023

(54) METHYL 2-ALLYL-1 -METHYL-3-OXOINDOLINE-2-CARBOXYLATE AND 9A-ALLYL-1,2,3,9A-TETRAHYDRO- 9H-PYRROLO[1,2-A]INDOL-9-ONE DERIVATIVES AND RELATED COMPOUNDS FOR USE AS FLUORESCENT MARKERS FOR LABELLING OF DRUGS, AMINO ACIDS AN PROTEINS

(71) Applicant: UNIVERSITY OF SALFORD, Salford (GB)

(72) Inventor: Joseph Bernard SWEENEY, Lancaster, Lancashire (GB)

(21) Appl. No.: 17/784,161

(22) PCT Filed: Dec. 10, 2020
(86) PCT No.: PCT/GB2020/053167

§ 371 Date: Jun. 10, 2022

(30) Foreign Application Priority Data
Dec. 10, 2019 (GB) 1918128.8

(51) Int. Cl. C07D 209/42; C07D 487/04; G01N 33/58

(52) U.S. Cl. CPC C07D 209/42; C07D 487/04; G01N 33/582

(57) ABSTRACT

Compounds of formulae (3) and (I) for use as fluorescent markers for labelling an amine containing target drug, an amino acid or a protein are disclosed. Exemplary compounds are e.g. (II) (III) (IV).

The present invention relates to a new class of fluorescent oxindoles and a novel method to produce such heterocycles. The fluorescent oxindoles are generally 3-oxindoles such as 2,2-di-substituted 3-oxindoles. There is also the use of such compounds as fluorescent markers and a method of tagging molecules such as amines using such compounds. According to a further aspect of the present invention, there is provided a method to prepare chemical matter for use as in-cell imaging agents, and a method capable of being used to prepare fluorescent amino acids for incorporation into proteins.

### BACKGROUND TO THE DISCLOSURE

Indole is one of the most important structural units of natural products and pharmaceuticals and consists of a benzene ring fused to a 5 membered N containing pyrrole ring. The related 2- and 3-oxoindolines (‘oxindoles’) are also important structural units.

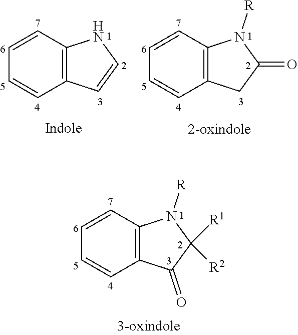

Due to the presence of the oxindole motif in a diverse range of bioactive small molecules, synthetic routes to oxindoles have attracted intense interest. Though there are many elegant methods reported to access 2-oxindoles, there is a paucity of methodology for the direct synthesis of 3-oxindoles; this is especially true for 2,2-disubstitued oxindoles. These compounds have many interesting and useful properties, such as pronounced biological activity (as exemplified by austamide, mitrogynine pseudoindoxyl which has a 20-fold higher potency than morphine at μ-opioid receptors and 35-fold higher at δ-opioid receptors). Known methods of synthesising substituted 3-oxindoles such as 2,2 di-substituted 3-oxindoles tend to have a low associated yield.

In addition to biomodulating activity, 2,2-disubstituted 3-oxindoles have also been reported as useful and practical imaging agents, due to their fluorescence properties; for instance, Lipid Green has been used in whole organisms (zebrafish) to image lipid pools.

### SUMMARY OF THE INVENTION

According to a first aspect of the present invention there is provided new 3-oxindoles, in particular, di-substituted 3-oxindoles, such as 2,2 di-substituted 3-oxindoles.

According to a first aspect of the present invention, there is provided a compound of the formula (I):

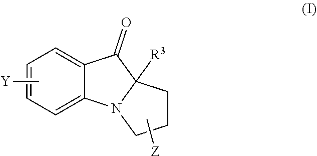

wherein
   R3 represents a 2-alkenyl or a 1-allenyl group;
   each Y group independently represents H; a group presenting heteroatoms, such as oxygen, or halogen, in particular an alkoxy group, a group containing fluorine or chlorine; or an optionally substituted hydrocarbyl group, such as an optionally substituted alkyl group, an optionally substituted (CH₂)K-carbocyclyl group or an optionally substituted (CH₂)K-aryl group where K represents an integer from 0 to 6, generally 0, 1, 2, or 3;
each Z group independently represents H, halogen, an optionally substituted hydrocarbyl group (such as an optionally substituted alkyl group), or a group presenting heteroatoms, in particular oxygen, nitrogen or halogen, such as an alkoxy group, or a group containing or consisting of fluorine or iodine.

Each Z group is generally monovalent. Each Z group is linked to the carbocyclyl group through a single covalent bond.

R3 generally represents an optionally substituted hydrocarbyl group including an alkenyl group, and/or an allenyl group (suitably an alkenyl group, or an allenyl group), a carbocycle group including a ring moiety and one or more alkenyl groups, or a carbocycle group including a ring moiety comprising one or more double bonds between adjacent alicyclic carbon atoms.

According to one embodiment, R3 represents a group including at least one double bond, generally R3 represents an alkenyl group, an allenyl group, or a carbocycle group including a ring moiety and one or more alkenyl groups;

each Y group independently represents H, an optionally substituted hydrocarbyl group, or an alkoxy group;

each Z group independently represents H, an optionally substituted hydrocarbyl group, or an alkoxy group.

According to a further aspect of the present invention, there is provided a compound according to formula (Ia):

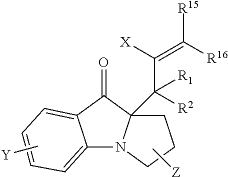

Wherein Y and Z are as defined above;
   R1, R2, R15, R16 and X independently represent H, an optionally substituted hydrocarbyl group (such as an alkyl group), where two or more of the R1, R2, R15 and R16 groups may combine to form a carbocyclyl group.

According to one embodiment, R1, R2, R15 and R16 independently represent H, an optionally substituted hydrocarbyl group, or an alkoxy group, where one or more of the R1, R2, R15 and R16 groups may combine to form an aryl group;

X represents H or an optionally substituted hydrocarbyl group such as an alkyl group.

According to a further aspect of the present invention, there is provided a compound according to formula (Ib):

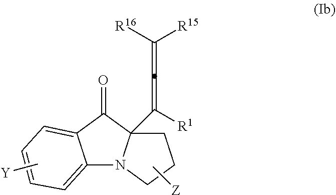

wherein Y and Z are as defined above;
R1, R15 and R16 independently represent H, an optionally substituted hydrocarbyl group (such as an optionally substituted alkyl group), where two or more of the R1, R15 and R16 groups may combine to form a carbocyclyl group.

According to a further aspect of the present invention, there is provided a compound according to formula (3):

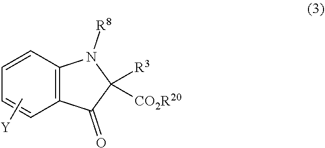

wherein
   R3 represents a 2-alkenyl or a 1-allenyl group;
   each Y group independently represents H; a group presenting heteroatoms such as oxygen or halogen, in particular an alkoxy group, a group containing fluorine or chlorine; or an optionally substituted hydrocarbyl group, such as an optionally substituted alkyl group, an optionally substituted —(CH₂)K-carbocycyl group, an optionally substituted —(CH₂)K-aryl group, or an optionally substituted—(CH₂)K-heteroaryl group, where K represents an integer from 0 to 6, generally 0, 1, 2, or 3;
R8 represents an optionally substituted hydrocarbyl group, in particular an optionally substituted alkyl group, an optionally substituted alkynyl group, an optionally substituted —(CH₂)L-carbocycyl group an optionally substituted (CH₂)L-aryl group, or an optionally substituted (CH₂)L-heteroaryl group where L represents an integer from 1 to 15, suitably from 1 to 6, generally 1, 2, or 3;
R20 represents an alkyl group, in particular C1 to 6 alkyl group.

Generally wherein R3 represents an optionally substituted hydrocarbyl group including an alkenyl group, and/or an allenyl group (suitably an alkenyl group, or an allenyl group), a carbocycle group including a ring moiety and one or more alkenyl groups, or a carbocycle group including a ring moiety comprising one or more double bonds between adjacent alicyclic carbon atoms.

According to one embodiment, R3 represents a group including at least one double bond, generally represents an alkenyl group, an allenyl group, or a carbocycle group including a ring moiety and one or more alkenyl groups;

each Y group independently represents H, an optionally substituted hydrocarbyl group, or an alkoxy group;

R8 represents H, or an optionally substituted hydrocarbyl group;

R20 represents an alkyl group.

According to a further aspect of the present invention, there is provided a compound according to formula (3a)

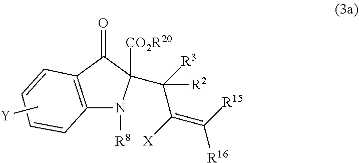

wherein
   R1, R2, R15, R16 and X independently represent H, or an optionally substituted hydrocarbyl group (in particular an alkyl group, such as a C1 to 6 alkyl group), where two or more of the R1, R2, R15 and R16 groups may combine to form a carbocyclyl group;

According to one embodiment, R1, R2, R15 and R16 may independently represent H, an optionally substituted hydrocarbyl group, or an alkoxy group, where one or more of the R1, R2, R15 and R16 groups may combine to form an aryl group;

X represents H or an optionally substituted hydrocarbyl group an alkyl group.

According to one aspect of the present invention, there is provided compounds according to formula (3b):

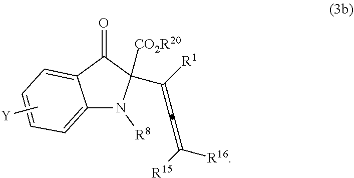

wherein R1, R15 and R16 independently represent H, or an optionally substituted hydrocarbyl group, where two or more of the R1, R15 and R16 groups may combine to form a carbocyclyl group.

The compounds of the present invention are generally fluorescent, and tend to possess physical properties suitable for fluorescent labelling. The compounds of the present invention are generally non-charged. Typically, the compounds disclosed herein comprise a chiral centre. Suitably, the compounds of the present invention are non-toxic to a human or animal body. Typically, the compounds have a number average molecular weight of 500 g/mol or less.

The present invention provides bioactive small molecules and low molecular weight fluorescent oxindole chemical probes, in particular chiral fluorescent oxindole materials, for use as functional materials for a range of biological purposes.

According to a further aspect of the present invention, there is provided a method of synthesising 3-oxindoles, in particular, di-substituted 3-oxindoles such as 2,2 di-substituted 3-oxindoles.

The method of synthesis tends to have a greater associated yield than known methods of synthesising 3-oxindoles, in particular, 2,2 di-substituted 3-oxindoles. Where the method is for the synthesis of 2,2 di-substituted 3-oxindoles, the associated yield is generally at least 40%, typically at least 44%, suitably at least 50% .

According to a further aspect of the present invention, there is provided the use of the compounds disclosed herein as fluorescent labels.

The present invention provides non-charged fluorescent labels. This is in stark contrast to the vast majority of marketed fluorescent imaging agents, which are either positively or negatively charged or zwitterionic (overall neutral, but containing both positive and negative charge). The fluorescent labels of the present invention will accordingly tend to accrue in non-charged cellular regions, such as membranes.

There is also provided a method of labelling an amine using the compounds disclosed herein. According to a further aspect of the present invention, there is provided a labelled amine compound.

The method offers a novel way to introduce a fluorescent tag into chemical molecules. More than half of all marketed medicines are believed to contain amine functionality. The methods and compounds of the present invention also allow the production of fluorescently labelled polymers and soft materials.

Where appropriate, teaching relating to any aspect or embodiment may relate to any other embodiment.

Throughout the Application, where a compound or composition is described as having, including, or comprising specific components, or where methods are described as having, including, or comprising specific method steps, it is contemplated that compound or composition of the present teachings also consist essentially of, or consist of, the recited components, and that the methods of the present teachings also consist essentially of, or consist of, the recited method steps.

In the Application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a device, or a method described herein can be combined in a variety of ways without departing from the spirit and scope of the present teachings, whether explicit or implicit herein.

The use of the terms “include,” “includes”, “including,”, “comprise”, “comprises” “comprising”, “have,” “has,” or “having” should be generally understood as open-ended and non-limiting unless specifically stated otherwise.

The use of the singular herein includes the plural (and vice versa) unless specifically stated otherwise. All numerical values provided incorporate 10% less than and 10% more than the numerical value provided.

In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present teachings remain operable. Moreover, two or more steps or actions may be conducted simultaneously.

Features, integers, characteristics, or groups described in conjunction with a particular aspect, embodiment or example of the invention are to be understood to be applicable to any other aspect, embodiment or example described herein unless incompatible therewith.

### DEFINITIONS

### Hydrocarbyl

The term “hydrocarbyl” as used herein includes reference to moieties consisting exclusively of hydrogen and carbon atoms; such a moiety may comprise an aliphatic and/or an aromatic moiety. In some embodiments, the moiety may comprise up to 100 carbon atoms, suitably up to 75 carbon atoms, typically up to 50 carbon atoms, generally 10 to 40 carbon atoms.

Examples of hydrocarbyl groups include C₁₋₆ alkyl (e.g. C₁, C_{2,} C₃ or C₄ alkyl, for example methyl, ethyl, propyl, isopropyl, n-butyl, sec-butyl or tert-butyl);; cycloalkyl (e.g. cyclopropyl, cyclobutyl, cyclopentyl or cyclohexyl); alkenyl (e.g. 2-butenyl); alkynyl (e.g. 2-butynyl); aryl (e.g. phenyl, benzyl, naphthyl) and the like. A hydrocarbyl may be saturated or unsaturated and may include alkyl, alkenyl, alkynyl, carbocycle, for example aryl groups. A hydrocarbyl group or portion may be straight chain or branched, and may be substituted or unsubstituted.

### Alkyl

The term “alkyl” as used herein includes reference to a straight or branched chain alkyl moiety. In some embodiments, the moiety may comprise up to 100 carbon atoms, suitably up to 75 carbon atoms, typically up to 50 carbon atoms, generally 1 to 40 carbon atoms. According to some embodiments, the moiety may have 1 to 15 carbon atoms, generally 1 to 10 carbon atoms, suitably 1, 2, 3, 4, 5 or 6 carbon atoms. This term includes reference to groups such as methyl, ethyl, propyl (n-propyl or isopropyl), butyl (n-butyl, iso-butyl, sec-butyl or tert-butyl), pentyl, hexyl, dimethyl, diethyl, dipropyl, dibutyl and the like. The term “small alkyl group” refers to an alkyl group having a carbon backbone of 1 to 6 carbon atoms, typically 1 to 4 carbon atoms.

### Alkenyl

The term “alkenyl” as used herein includes reference to a straight or branched chain alkyl moiety and having at least one double bond, of either E or Z stereochemistry where applicable. This term includes reference to straight or branched chain alkyl moieties. In some embodiments, the moiety may comprise up to 100 carbon atoms, suitably up to 75 carbon atoms, typically up to 50 carbon atoms, generally 2 to 40 carbon atoms. According to some embodiments, the moiety may have 2 to 10 carbon atom, including 2, 3, 4, 5 or 6 carbon atoms such as ethenyl, 2-propenyl, 1-butenyl, 2-butenyl, 3-butenyl, 1-pentenyl, 2-pentenyl, 3-pentenyl, 4-pentenyl, 1-hexenyl, 2-hexenyl 3-hexenyl , 4-hexenyl, 5-hexenyl and the like. The term “small alkenyl group” refers to an alkenyl group having a carbon backbone of 3 to 6 carbon atoms, typically 3 to 4 carbon atoms

### Alkynyl

The term “alkynyl” as used herein includes reference to a straight or branched chain alkyl moiety having at least one triple bond. This term includes reference to straight or branched chain alkyl moieties. In some embodiments, the moiety may comprise up to 100 carbon atoms, suitably up to 75 carbon atoms, typically up to 50 carbon atoms, generally 2 to 40 carbon atoms. According to some embodiments, the moiety may have 2 to 10 carbon atom, including 2, 3, 4, 5 or 6 carbon atoms such as ethynyl, 1-propynyl, 2-propynyl, 1-butynyl, 2-butynyl, 3-butynyl, 1-pentynyl, 2-pentynyl, 3-pentynyl, 4-pentenyl, 1-hexynyl, 2-hexynyl, 3-hexynyl, 4-hexanyl, 5-hexanyl and the like. The term “small alkynyl group” refers to an alkynyl group having a carbon backbone of 2 to 6 carbon atoms, typically 3 to 4 carbon atoms.

### Allenyl

The term “allenyl” is used to refer to a straight or branched chain alkyl moiety having two directly adjacent double bonds. One carbon atom of an allene has double bonds with each of its two adjacent carbon centres. This term includes reference to straight or branched chain alkyl moieties. In some embodiments, the moiety may comprise up to 100 carbon atoms, suitably up to 75 carbon atoms, typically up to 50 carbon atoms, generally 3 to 40 carbon atoms. According to some embodiments, the moiety may have 3 to 10 carbon atoms, including 3, 4, 5 or 6 carbon atoms.

### Alkoxy

An alkoxy group includes an alkyl group singularly bonded to oxygen.

### Carbocycle T

he term “carbocycle” as used herein includes reference to a saturated (e.g. cycloalkyl) or unsaturated (e.g. aryl) ring moiety. In some embodiments, the moiety may comprise up to 100 carbon atoms, suitably up to 75 carbon atoms, typically up to 50 carbon atoms, generally 5 to 30 carbon atoms. In some embodiments, the moiety has 3, 4, 5, 6, 7, 8, 9, 10, 11, 12, 13, 14, 15 or 16 carbon ring atoms. In particular, carbocycle includes a 3- to 10-membered ring or ring system and, in particular, a 5- or 6-membered ring, which may be saturated or unsaturated. A carbocyclic moiety is, for example, selected from cyclopropyl, cyclobutyl, cyclopentyl, cyclohexyl, cyclopentenyl, cyclohexenyl, norbornyl, bicyclo[2.2.2]octyl, phenyl, benzyl, naphthyl, fluorenyl, azulenyl, indenyl, anthryl and the like.

### Aryl

The term “aryl” as used herein includes reference to an aromatic ring system. In some embodiments, the ring system may comprise up to 100 carbon atoms, suitably up to 75 carbon atoms, typically up to 50 carbon atoms, generally 5 to 40 carbon atoms. In some embodiments, the ring system comprises 6, 7, 8, 9, 10, 11, 12, 13, 14, 15 or 16 ring carbon atoms. Aryl is often phenyl but may be a polycyclic ring system, having two or more rings, at least one of which is aromatic. This term includes reference to groups such as phenyl, naphthyl, fluorenyl, azulenyl, indenyl, anthryl and the like.

### Heteroatom

The term heteroatom refers to any atom except C and H. Common heteroatoms include oxygen, halogen, nitrogen.

### DETAILED DESCRIPTION

According to a first aspect of the present invention there is provided new 3-oxindoles, in particular, di-substituted 3-oxindoles such as 2,2 di-substituted 3-oxindoles.

According to an aspect of the present invention, there is provided a 3-oxindole, generally having the formula (3):

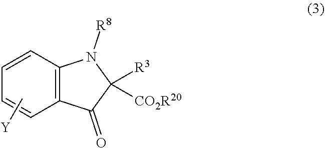

According to a further aspect of the invention, there is provided a chiral 2,2-disubstituted 3-oxindole of the general formula (I):

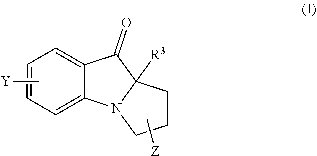

Wherein R3, R8, R20, Y, and Z are as defined above.

R3 represents a 2-alkenyl or a 1-allenyl group.

According to one embodiment, R3 generally represents an optionally substituted straight chain 2-alkenyl group, an optionally substituted straight chain allenyl group, a carbocycle group including a ring moiety (including a saturated or unsaturated ring moiety) and one or more alkenyl groups or a carbocycle group including a ring moiety comprising one or more double bonds between adjacent alicyclic carbon atoms, such as an optionally substituted—(CH₂)G-carbocyclyl group, where G represents an integer from 1 to 6, generally 1, 2, or 3;

Each Y group may independently represent H; a group presenting heteroatoms such as oxygen or halogen, in particular an alkoxy group, a group containing fluorine or chlorine; or an optionally substituted hydrocarbyl group, such as an optionally substituted alkyl group, an optionally substituted —(CH₂)K-carbocycyl group, an optionally substituted —(CH₂)K-aryl group.

Each Y group generally independently represents H, an alkoxy group, fluorine, chlorine, an optionally substituted alkyl group, an optionally substituted (CH₂)K-carbocyclyl group or an optionally substituted (CH₂)K-aryl group where K represents an integer from 0 to 6, generally 0, 1, 2, or 3.

Typically, R8 represents an optionally substituted C1 to 6 straight or branched chain alkyl group, an optionally substituted alkynyl group, an optionally substituted —(CH₂)L-carbocyclyl group, an optionally substituted (CH₂)L-aryl group, or an optionally substituted (CH₂)L-heteroaryl group, where L represents an integer from 1 to 15, suitably from 1 to 6, generally 1, 2, or 3. Generally the carbocyclyl, aryl or heteroaryl group comprises one or more 5 to 7 carbon ring atoms, 5 or 6.

According to one embodiment, R8 represents an optionally substituted C1 to 6 straight or branched chain alkyl group, an optionally substituted alkynyl group, an optionally substituted —(CH₂)L-carbocyclyl group, or an optionally substituted (CH₂)L-aryl group, where L represents an integer from 1 to 15, suitably from 1 to 6, generally 1, 2, or 3. Generally the carbocyclyl, or aryl group comprises one or more 5 to 7 carbon ring atoms, 5 or 6.

R20 generally represents methyl, ethyl or propyl.

Each Z group generally independently represents H, an optionally substituted alkyl group an alkoxy group, fluorine, or a group containing nitrogen or iodine. In particular, Z is generally H, a small alkyl group or a halogen containing group.

According to one embodiment, Z represents a hydrocarbyl group substituted with one or more oxygen, halogen or nitrogen group, generally a C1 to 6 hydrocarbyl group substituted with one or more oxygen, fluorine, iodine, or nitrogen group.

Each Z group is typically monovalent. Each Z group is typically linked to the carbocyclyl group through a single covalent bond.

According to one embodiment, R3 represents a 2-alkenyl group including 3 to 100 carbon atoms, generally 3 to 50 carbon atoms, typically 3 to 25 carbon atoms, suitably 3 to 7 carbon atoms. Alternatively, R3 represents a 1-allenyl group including 3 to 100 carbon atoms, generally 3 to 50 carbon atoms, typically 3 to 25 carbon atoms, suitably 3 to 5 carbon atoms. According to one embodiment, R3 represents 2-propenyl or a propadiene group.

R3 may represent a 2-alkenyl group including 3 to 7 carbon atoms, a 1-allenyl group including 3 to 5 carbon atoms, a carbocycle group including a ring moiety having 5 to 7 carbon ring atoms and one or more ethenyl groups, or a carbocycle group including a ring moiety having 5 to 7 carbon ring atoms comprising one or more double bonds between adjacent alicyclic carbon atoms.

According to one embodiment, R3 represents a 2-alkenyl group, generally a straight or branched chain alkenyl group including 3 to 7 carbon atoms. Typically R3 represents a straight chain 2-alkenyl group including 3 to 6 carbon atoms, suitably 3 to 5 carbon atoms, generally 3 carbon atoms. According to one embodiment, R3 represents a propenyl group, generally 2-propenyl. Alternatively, R3 may represent a branched chain 2-alkenyl group including 4 to 7 carbon atoms, suitably 4 or 5 carbon atoms. R3 may represent a branched propylene or a branched butylene groups, typically wherein the hydrocarbon backbone of the alkylene group is branched with one or more methyl or ethyl groups, suitably one or two methyl groups.

According to one embodiment, R3 represents an unbranched 2-alkenyl group including 3 or 4 carbon atoms.

According to one embodiment, R3 represents a 1-allenyl group, generally a 1-allenyl group including 3 to 5 carbon atoms, typically, a propadiene group.

According to a further embodiment, R3 represents a carbocycle group including a ring moiety and one or more alkenyl groups, suitably one or two alkenyl groups, typically one or two ethenyl groups. The ring moiety is generally saturated having 5 to 7 carbon ring atoms, suitably 6 carbon ring atoms. R3 may suitably be a cycloalkyl comprising one or more ethenyl groups. R3 may represent an optionally substituted —(CH₂)G-carbocyclyl group, where G represents an integer from 1 to 6, generally 1, 2, or 3.

Preferably R3 represents an unbranched 2-alkenyl group including 3 or 4 carbon atoms, such as 2-propenyl or an allenyl group such as a propadiene group.

Suitably R3 represents an 2-alkene, 1-allene, such as a carbocyclyl group comprising an unsaturated ring moiety and one or more alkene groups.

According to one embodiment, R3 represents a 2-alkenyl group including 2 to 6 carbon atoms, a 1-allenyl group including 3 to 5 carbon atoms, or a carbocycle group including a ring moiety having 5 to 7 carbon ring atoms and one or more ethenyl groups.

Generally, R3 does not include any aryl groups.

Each Y group independently represents H, an optionally substituted hydrocarbyl group, a group comprising heteroatoms (such as oxygen, fluorine, or chlorine), or an alkoxy group. Typically, Y represents H, or alkyl, or alkoxy, or aryl but could also represent or include halogen heteroatoms. Typically an alkyl group can include 1 to 6 carbon atoms; alkoxy generally having the structure O-alkyl where “alkyl” typically represents a C1 to 6 alkyl group, generally methyl, ethyl or propyl. Suitably, each Y group independently represents H, an alkoxy group, or an optionally substituted hydrocarbyl group, such as an optionally substituted —(CH₂)K-carbocycyl group, an optionally substituted —(CH₂)K-aryl group, or an optionally substituted —(CH₂)K-heteroaryl group where K represents an integer from 0 to 6, generally 0, 1, 2, or 3.

Generally, Y is an alkyl group (suitably a C1 to 6 alkyl group, generally methyl), an alkoxy group, (in particular —O—-C1 to C6 group, suitably —O—Me), H, fluorine, chlorine, or (CH₂)K-aryl group, (such as (CH₂)K-phenyl group) where K represents an integer from 0 to 6, generally 0). Typically one Y group is present, although in some embodiments two Y groups may be preferred, in particular where Y represents an alkoxy group.

Y is generally a monovalent group. Y is generally linked to the carbocyclyl group through a single covalent bond. However, other alternatives are envisaged, for instance a fused ring system.

Where Y represents a hydrocarbyl group, it is typically a substituted or unsubstituted alkyl group such as a C_{1 to 6} alkyl group. The alkyl group may be straight chain or branched. The alkyl group may include one or more heteroatoms such as nitrogen, oxygen, halogen (in particular, fluorine or chlorine). Alternatively, or additionally, the alkyl group may be substituted with one or more hydrocarbyl group and or one or more halogen group. Generally, Y represents H, methyl, ethyl or propyl, typically methyl or ethyl. Alternatively, where Y represents a hydrocarbyl group, it may be a carbocycle, in particular, an aryl group. Y may represent a polycyclic ring system. According to one embodiment, Y may represent a phenyl group.

According to one embodiment, Y represents H, a straight chain alkyl group including 1 to 6 carbon atoms, a substituted or unsubstituted C5 to 7 aryl group or an alkoxy group having the structure O—C1 to 6 alkyl group. Y may represent H, methyl, methoxy or a phenyl group.

Typically, Y is selected from the group consisting of H, methoxy (MeO), methyl (Me), ethyl (Et), aryl (Ar) (naphthalene derivatives) a heteroatom such as F, Cl, N, 0 and/ or the like. Suitably, Y represent H, MeO, Me, (CH₂)K-phenyl group) where K represents an integer from 0 to 1, generally 1. According to one embodiment, Y represents H.

According to a further embodiment, Y may represent an aryl group such as an optionally substituted (CH₂)K-aryl group, where K represents an integer from 0 to 6, typically 0, 1, 2, or 3. Y may represent a substituted or unsubstituted C5 to 7 aryl group. Generally, Y represents phenyl.

According to one embodiment, Y represents an alkoxy group, generally having the structure O-alkyl where “alkyl” typically represents a C1 to 6 alkyl group, generally methyl, ethyl or propyl. According to one embodiment, Y represents methoxy.

According to one embodiment, Y represents H, methyl, methoxy or a phenyl group.

The compound of formula (I) and the compounds of formula (3) may include more than one Y substituent which does not represent H, generally 1 or 2 of such Y substituents. Where the compounds of formula (I) or the compounds of formula (3) include more than one Y substituent which does not represent H, each is independently selected and may be the same or different. All positions of the benzene ring of formula (I) or formula (3) may be substituted. According to one embodiment, positions 4, 5 and/or 6 positions of the benzene ring are substituted.

Generally, Y is an alkyl group (suitably a C1 to 6 alkyl group, generally methyl), an alkoxy group, (in particular —O—C1 to C6 group, suitably —O—Me), H or (CH₂)K-aryl group, (such as (CH₂)K-phenyl group) where K represents an integer from 0 to 6, generally 0). Typically one Y group is present, although in some embodiments two Y groups may be preferred, in particular where Y represents an alkoxy group.

Generally, Y represents H, methoxy or methyl.

Typically, Y represents H.

Each Z group is generally monovalent, attaching to the ring group of formula (I) via a single covalent bond.

Each Z group independently represents H, an optionally substituted hydrocarbyl group, or a group comprising heteroatoms such as an alkoxy group.

Generally Z represents H.

According to one embodiment, Z does not represent H and the compound of formula (I) may include more than one Z substituent, generally 1 or 2 Z substituents. Where the compound of formula (I) includes more than one Z substituent, each is independently selected and may be the same or different.

Generally, Z represents H or a hydrocarbyl group, typically having 1 to 6 carbon atoms. Suitably where Z is a hydrocarbyl group it represents an alkyl group, in particular a methyl, ethyl, propyl or butyl group, suitably a methyl group.

According to one embodiment, Z represents a group presenting heteroatoms, in particular oxygen, nitrogen or halogen, such as an alkoxy group, or a group containing or consisting of fluorine or iodine.

According to one embodiment, the compounds of the invention are of the formula (Ia)

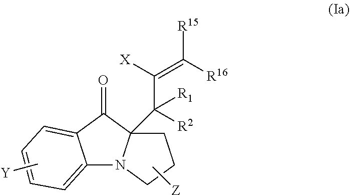

Wherein Y and Z are as defined above.

Wherein R1, R2, R15, R16 and X independently represent H, an optionally substituted hydrocarbyl group (typically an alkyl, alkenyl, aryl, carbocycle, or alkynyl group). Typically, R1, R2, R15, R16 and X independently represent H, an alkyl group, or an aryl group, where two or more of the R1, R2, R15, R16 and X groups may combine to form a carbocyclyl group. Suitably R1, R2, R15, R16 and X independently represent H or an alkyl group, in particular C1 to C6 alkyl group (generally methyl, ethyl or propyl, suitably methyl).

Generally R1, R2, R15, R16 and X independently represent H or methyl.

According to one embodiment, R1 and R2 may independently represent a straight or branched chain alkyl moiety, which may be substituted or unsubstituted. Suitable substituents include hydrocarbyl groups. Typically R1 and R2 independently represent a straight or branched chain alkyl moiety having 1 to 10 carbon atoms, suitably 1, 2, 3, 4, 5 or 6 carbon atoms. The term “alkyl” includes reference to groups such as methyl, ethyl, propyl (n-propyl or isopropyl), butyl (n-butyl, iso-butyl, sec-butyl or tert-butyl), pentyl, hexyl, dimethyl, diethyl, dipropyl, dibutyl and the like. Where R1 and R2 independently represent an alkyl group, one or both of R1 and R2 may be in the form of a straight or branched chain alkyl group, typically having 1 to 6 carbon atoms, suitably having 1, 2, 3 or 4 carbon atoms.

According to one embodiment, one or both of R1 and R2 may represent H. Suitably, both of R1 and R2 represent H.

Generally, R15 and R16 represent H. Alternatively R15 and R16 independently represent an alkyl, alkylene or unsaturated carbocycle group, in particular a C_{1 to 6} alkyl group. Generally, R15 and R16 independently represent a H or a straight chain C_{1 to 6} alkyl; suitably methyl, ethyl, propyl or butyl group, typically a methyl or ethyl group, suitably R15 and R16 represent H or Me.

Generally represents H or an optionally substituted hydrocarbyl group, suitably an optionally substituted alkyl group, generally an unsubstituted alkyl group. In particular, X generally represents H or a C₁ to C6 alkyl group; suitably, H, methyl, ethyl or propyl; generally H or methyl.

X may represent H or an alkyl group. Generally, X represents H. Alternatively X represents an alkyl group, in particular a C_{1 to 6} alkyl group. Generally, X represents a straight chain C_{1 to 6} alkyl; suitably methyl, ethyl, propyl or butyl group, typically a methyl or ethyl group, suitably a methyl group.

Generally, X represents H or methyl.

Typically, R15, R16 and Z represent H. X generally represents H.

Exemplary structures are provided below where R1, R2, X, Y and Z are as defined above. According to one embodiment, Y and Z represent hydrogen for the exemplary structures below.

According to a further embodiment, any of the exemplary structures below may have one or more Y group and/or one or more Z group that does not represent H. Suitably Y does not represent H and the compounds of the exemplary structure below have one or two Y group substituents. The Y and/or Z substituents may be present at any position on the associated carbocycle group.

According to one embodiment, the compounds of the invention are of the formula (Ib)

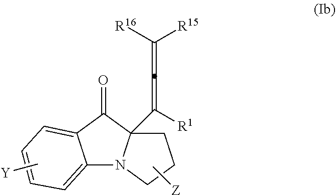

Wherein Y, Z, R1, R15 and R16 are as defined herein.

In one embodiment of the invention, there is provided a 2,2-disubstituted 3-oxindole compound having the structure of formula (II):

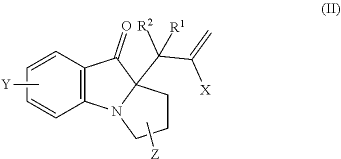

Wherein Y, Z, R1, R2, and X are as defined herein.

According to one embodiment, X represents hydrogen and both of R1 and R2 represent hydrogen.

Exemplary structures are provided below where Y, Z, X, R8, R15, R16 and R20 are as defined herein.

According to one embodiment of the invention, there is provided a 2,2-disubstituted 3-oxindole compound having the structure of formula (III):

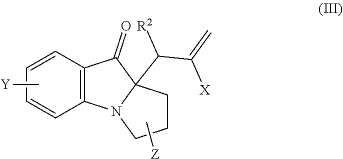

According to one embodiment, X represents H and R2 represents an alkyl group, suitably a small alkyl group such as methyl. R1 is not shown and represents H.

In one embodiment of the invention 2,2-disubstituted 3-oxindole has the structure of formula (IV):

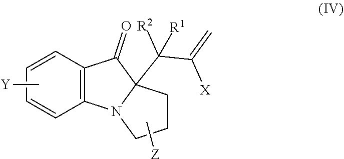

According to one embodiment, X represents H and R1 and R2 both independently represent an alkyl group, preferably a small alkyl group such as methyl.

According to one embodiment, X represents a small alkyl group such as methyl, and R1 and R2 both represent hydrogen.

In one embodiment of the invention 2,2-disubstituted 3-oxindole has the structure of formula (VI):

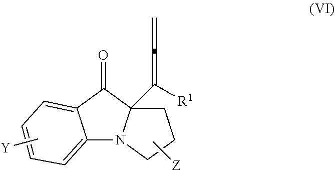

The compounds of formula (VI) may include one or more Y groups and/or one or more Z groups.

According to one embodiment, R1 represents H.

According to a further embodiment, R1 represents an alkyl group, in particular a small alkyl group such as methyl.

According to a further embodiment, R1 represents an alkyl group, in particular a small alkyl group such as ethyl.

As noted above, there is provided a 3-oxindole, generally having the structure of formula (3):

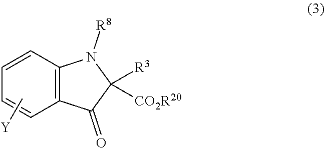

Wherein Y and R3, R8 and R20 are as defined above.

R8 represents H, or an optionally substituted hydrocarbyl group, such as an alkyl, alkylene, alkynyl or carbocycle group in particular an alkyl, alkene, or aryl group, suitably a C_{1 to 6} alkyl group, a C_{3 to 6} alkene group. Typically, R8 represents (CH₂)L-aryl group where L represents an integer from 1 to 3, such as (CH₂)-phenyl) or (CH₂)L-heteroaryl group where L represents an integer from 1 to 3. According to one embodiment, R8 represents methyl, ethyl, propyl, butyl or propargyl.

According to one embodiment, R8 represents a straight chain alkyl group having 1 to 6 carbon atoms, an aryl ring moiety having 5, 6 or 7 carbon ring atoms, or a straight chain alkylene group having a carbon backbone of 2 to 6 carbon atoms.

According to one embodiment, R8 represents an alkyl group, in particular a straight or branched chain alkyl group having a carbon backbone of 1 to 6 carbon atoms, typically 1 to 4 carbon atoms, generally a straight chain alkyl group having 1 to 6 carbon atoms; suitably 1 to 4 carbon atoms, suitably methyl, ethyl or propyl group. According to one embodiment, R8 represents a methyl group.

According to a further embodiment, R8 may represent a carbocycle group; generally, a saturated (e.g. cycloalkyl) or unsaturated (e.g. aryl) ring moiety having 5, 6 or 7 carbon ring atoms; typically, an aryl ring moiety having 5, 6 or 7 carbon ring atoms. According to one embodiment, R8 represents a benzyl, or a phenyl group. The aryl group may be attached via a hydrocarbyl group, in particular a straight chain alkyl group.

According to a further embodiment, R8 may represent an alkenyl group, generally a straight chain alkenyl group having a carbon backbone of 3 to 6 carbon atoms, typically 3 to 4 carbon atoms, suitably 2-propenyl.

According to a further embodiment, R8 may represent an alkynyl group, generally a straight chain alkynyl group having a carbon backbone of 3 to 6 carbon atoms, typically 3 to 4 carbon atoms, suitably, 2-propynyl, 2-butynyl. According to one embodiment, R8 represents a 2-propynyl group.

Preferably R8 represents an unbranched small alkyl group such as methyl.

Suitably R8 represents H or a hydrocarbyl group such as, a straight or branched alkyl, alkenyl, alkynyl or carbocyclyl group, such as an aromatic group (such as a benzyl group).

Generally R8 represents methyl, propargyl or (CH₂)-phenyl.

R20 represents an alkyl group, generally a small alkyl group suitably including C1 to 6 such as methyl, ethyl or propyl. Generally R20 represents methyl.

Typically, Y may be H, halogen, methoxy, methyl, or a phenyl group. According to one embodiment, Y may represent a halogen. Structures of formula 3 may have more than one Y substituent. Suitably, Y represents methoxy and structures of formula (3) includes two Y substituents.

According to one embodiment, Y represents H, R8 represents an unbranched small alkyl group such as methyl and R3 represents an unbranched alkenyl group including 3 or 4 carbon atoms, such as 2-propenyl.

According to one embodiment, structures of formula (3) includes one Y group and Y represents an alkoxy group, such as methoxy, R8 represents an unbranched small alkyl group such as methyl and R3 represents an unbranched alkenyl group including 3 or 4 carbon atoms, such as 2-propenyl. In such embodiments, the Y group may suitably be at position 4. Alternatively, the Y group may be at position 5. According to a further embodiment, the Y group may be provided at position 6.

According to one embodiment, structures of formula (3) includes one Y group and Y represents an alkyl group, in particular a small alkyl group such as methyl, R8 represents an unbranched small alkyl group such as methyl and R3 represents an unbranched alkenyl group including 3 or 4 carbon atoms, such as 2-propenyl. In such embodiments, the Y group may suitably be at position 4. Alternatively, the Y group may be at position 5. According to a further embodiment, the Y group may be provided at position 6.

According to one embodiment, structures of formula (3) includes one Y group and Y represents an aryl group such as phenyl, R8 represents an unbranched small alkyl group such as methyl and R3 represents an unbranched alkenyl group including 3 or 4 carbon atoms, such as 2-propenyl. In such embodiments the Y group may be attached to the benzene ring of structures of formula (3) at positions 4 and 5 to form a polycyclic ring system.

According to one embodiment, Y represents H, R8 represents an unbranched small alkyl group such as methyl and R3 represents an allenyl group including 3 to 5 carbon atoms, typically, a propadiene group.

According to one embodiment, structures of formula (3) includes one Y group and Y represents an alkoxy group, such as methoxy, R8 represents an unbranched small alkyl group such as methyl and R3 represents an allenyl group including 3 to 5 carbon atoms, typically, a propadiene group. In such embodiments, the Y group may suitably be at position 4. Alternatively, the Y group may be at position 5. According to a further embodiment, the Y group may be provided at position 6.

According to one embodiment, structures of formula (3) includes one Y group and Y represents an alkyl group, in particular a small alkyl group such as methyl, R8 represents an unbranched small alkyl group such as methyl and R3 represents an allenyl group including 3 to 5 carbon atoms, typically, a propadiene group. In such embodiments, the Y group may suitably be at position 4.

According to one embodiment, structures of formula (3) includes one Y group and Y represents an aryl group such as phenyl, R8 represents an unbranched small alkyl group such as methyl and R3 represents an allenyl group including 3 to 5 carbon atoms, typically, a propadiene group. In such embodiments the Y group may be attached to the benzene ring of structure (3) at positions 4 and 5 to form a polycyclic ring system. Alternatively, the Y group may be attached to the benzene ring of structures of formula (3) at positions 5 and 6 to form a polycyclic ring system.

According to one embodiment, structures of formula (3) includes two Y groups and each Y independently represents an alkoxy group, in particular a small alkoxy group such as methoxy, R8 represents an unbranched small alkyl group such as methyl and R3 represents an allenyl group including 3 to 5 carbon atoms, typically, a propadiene group. In such embodiments, the Y groups may suitably be at positions 4 and 5.

According to one embodiment, structures of formula (3) includes one Y group and Y represents an alkoxy group, such as methoxy, R8 represents an unbranched small alkyl group such as methyl and R3 represents a branched alkenyl group, for instance a branched propenyl or butenyl group, preferably a branched propenyl group. The branched alkenyl group of R3 is typically substituted with one or more small alkyl groups, for instance one or two methyl groups. In such embodiments, the Y group may suitably be at position 4.

According to one embodiment, Y represents H, R8 represents an unbranched small alkyl group such as methyl and R3 represents a 2-alkenyl or a 1-allenyl group, such as a cycloalkyl group comprising one or more alkenyl groups, suitably a cycloalkyl comprising an ethenyl group.

According to one embodiment, Y represents H, R8 represents an unbranched small alkynyl group such as 2-propynyl and R3 represents an allenyl group including 3 to 5 carbon atoms, typically, a propadiene group.

According to one embodiment, Y represents H, R8 represents an unbranched small alkynyl group such as 2-propynyl and R3 represents a small alkenyl group such as 2-propenyl. According to one embodiment, structures of formula (3) includes one Y group and Y represents an alkoxy group, such as methoxy, R8 represents a carbocycle group, in particular an aryl group such as benzene and R3 represents a small alkenyl group such as 2-propenyl.

According to one embodiment, the compound is of formula (3a):

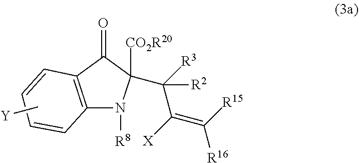

Where Y, X, R1, R2, R8, R15, R16 and R20 are as defined herein.

According to one embodiment, R15 and R16 independently represent H or a hydrocarbyl group, in particular an alkyl, alkylene or unsaturated carbocycle group. According to one embodiment, R15 and R16 independently represent H, or Me.

Generally, Y is an alkyl group (suitably a C1 to 6 alkyl group, generally methyl), an alkoxy group, (in particular —O—C1 to C6 group, suitably —O—Me), H or (CH₂)K-aryl group, (such as (CH₂)K-phenyl group) where K represents an integer from 0 to 6, generally 0). Typically one Y group is present, although in some embodiments two Y groups may be preferred, in particular where Y represents an alkoxy group.

Generally R8 represents an alkyl group (in particular a C1 to 6 alkyl group, in particular methyl), an alkynyl group (in particular a C3 to 6 alkynyl group, such as propynyl), or an aryl group (in particular, (CH₂)L-aryl group where L represents an integer from 1 to 3, such as (CH₂)-phenyl).

R20 generally represents methyl.

X generally represents H.

R1 and R2 generally independently represent H or C1 to 6 alkyl (in particular methyl).

R15 and R16 generally independently represent H or C1 to 6 alkyl (in particular methyl).

According to one embodiment, two or more of R1, R2, R15, R16 and X groups combine to form a carbocyclyl group.

According to one aspect of the present invention, there is provided compounds of formula (3b):

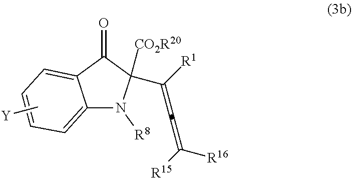

Where Y, R1, R8, R15, R16 and R20 are as defined herein.

Generally, Y is an alkyl group (suitably a C1 to 6 alkyl group, generally methyl), an alkoxy group, (in particular —O—C1 to C6 group, suitably —O—Me), H, halogen, or (CH₂)K-aryl group, (such as (CH₂)K-phenyl group) where K represents an integer from 0 to 6, generally 0). Suitably, Y is selected form the group consisting of C1 to 6 alkyl group, —O—C1 to C6 alkoxy group, H, (CH₂)K-phenyl group. Typically one Y group is present, although in some embodiments two Y groups may be preferred, in particular where Y represents an alkoxy group.

Generally R8 represents an alkyl group (in particular a C1 to 6 alkyl group, in particular methyl), an allyl group (in particular a C1 to C6 allyl group), an alkynyl group (in particular a C2 to 6 alkynyl group, such as propargyl), or an aryl group (in particular, (CH₂)L-aryl group where L represents an integer from 1 to 3, such as (CH₂)-phenyl). Preferably, R8 represents methyl.

R20 generally represents methyl.

R1 generally represents H.

R15 and R16 generally independently represent H or C 1 to 6 alkyl (in particular methyl). Suitably R15 and R16 independently represent H.

Illustrative, but non-limiting, examples of the compounds having structures of formula (3) of the present invention are shown below.

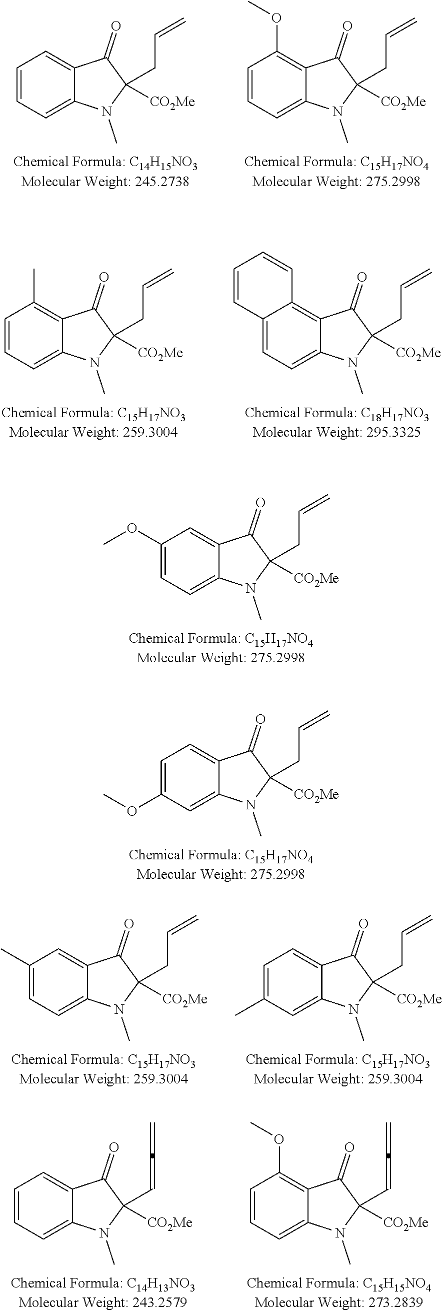

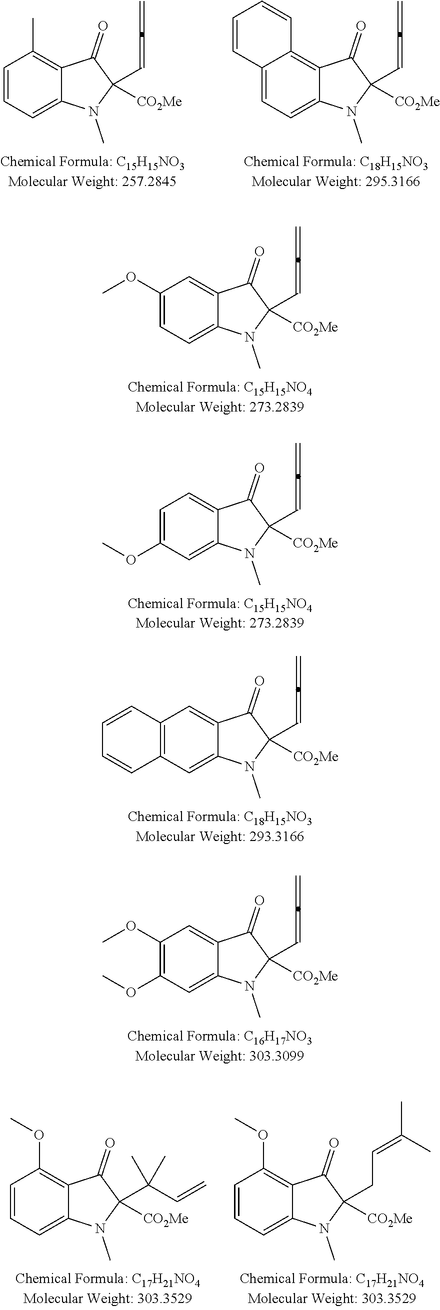

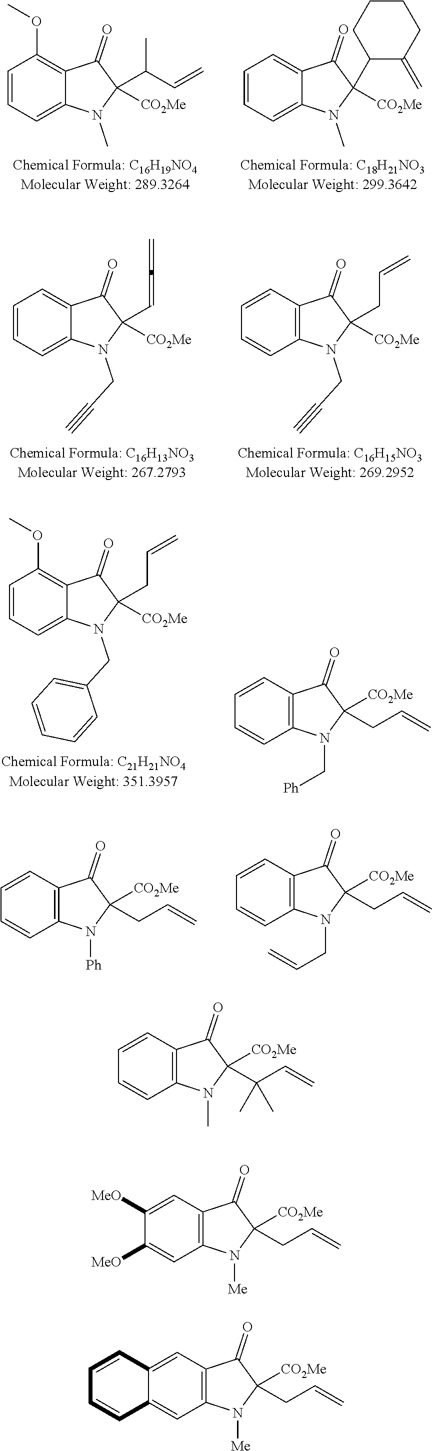

According to one embodiment, the compounds are of formula (3a), in particular having one of the structures below:

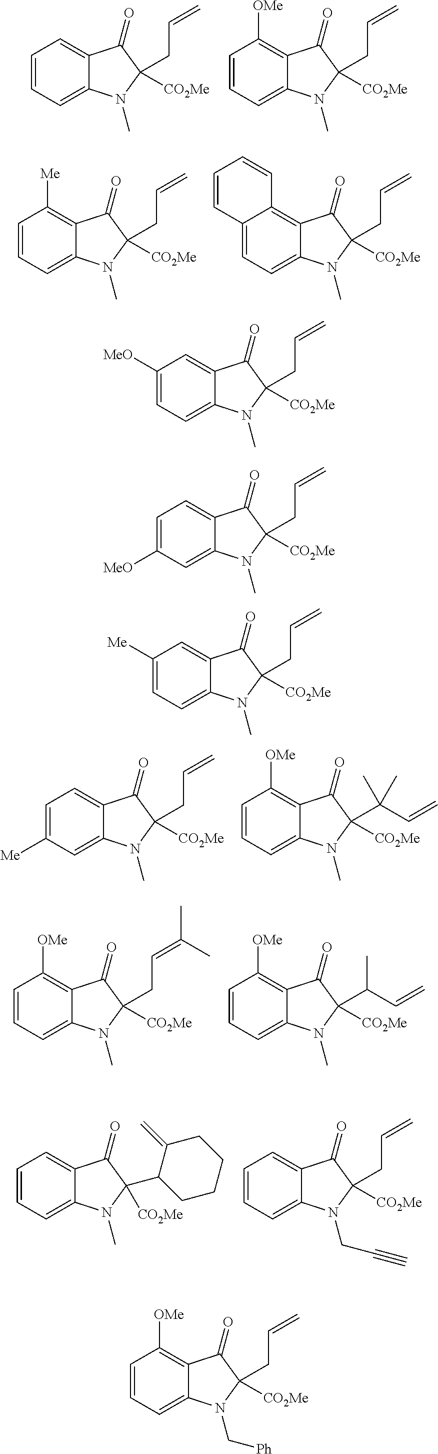

According to one embodiment, the compounds are of formula (3b), in particular having one of the structures below:

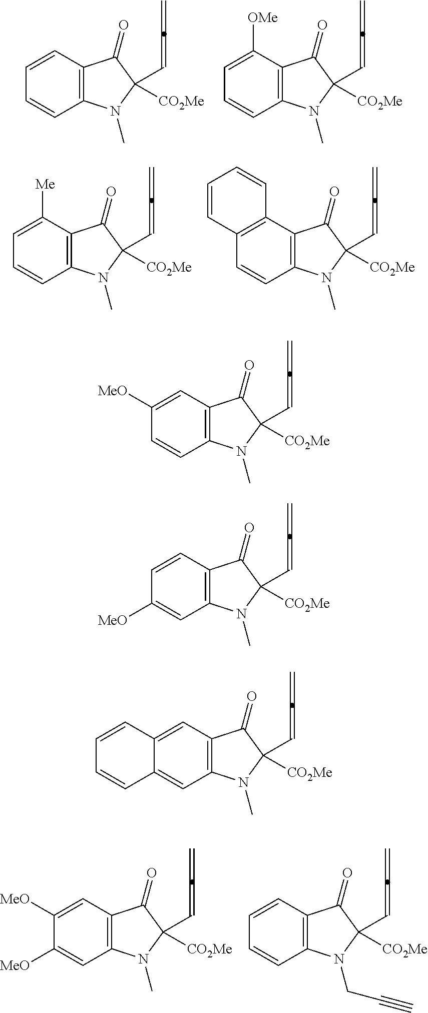

Typically the compounds of the invention are fluorescent and/or possess fluorescence properties.

Generally, the compounds of the present invention are uncharged, and this is in contrast to the majority of commercially available fluorescent imaging agents. This provides advantages when for use in non-charged environments, in particular non-charged cellular regions, such as membranes.

The compounds of the present invention generally have a number average molecular weight of less than 650 g/mol, typically less than 600 g/mol, suitably less than 500 g/mol, generally 100 to 500 g/mol, typically 200 to 300 g/mol.

The compounds disclosed herein generally have high biocompatibility and good photostability.

The present invention further provides a compound according to the invention which comprises the racemate, the S or the R enantiomer or a mixture thereof, of a compound according to the invention. Preferably, the compound is the S-enantiomer or the R-enantiomer.

Any mixtures of final products or intermediates obtained can be separated on the basis of the physico-chemical differences of the constituents, in a known manner, into the pure final products or intermediates, for example by chromatography, distillation, fractional crystallisation, or by the formation of a salt if appropriate or possible under the circumstances.

Compounds of the invention may be in the form of salts. In particular, the salts may be pharmaceutically acceptable salts. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., US, 1985, p. 1418, the disclosure of which is hereby incorporated by reference; see also Stahl et al, Eds, “Handbook of Pharmaceutical Salts Properties Selection and Use”, Verlag Helvetica Chimica Acta and Wiley-VCH, 2002.

The disclosure thus includes pharmaceutically-acceptable salts of the disclosed compounds wherein the parent compound is modified by making acid or base salts thereof

The compounds of the invention contain one or more asymmetric carbon atoms and may therefore exhibit enantiomerism or diastereoisomerism. All diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation, or by derivatisation, for example with a homochiral acid followed by separation of the diastereomeric derivatives by conventional means (e.g. HPLC, chromatography over silica). All stereoisomers are included within the scope of the disclosure. Where a single enantiomer or diasteromer is disclosed, the disclosure also covers the other enantiomers or diastereomers, and also racemates; in this regard, particular reference is made to the specific compounds listed herein.

Geometric isomers may also exist in the compounds of the present disclosure. The present disclosure contemplates the various geometric isomers and mixtures thereof resulting from the arrangement of substituents around a carbon-carbon double bond and designates such isomers as of the Z or E configuration.

The disclosure therefore includes all variant forms of the defined compounds, for example any tautomer or any pharmaceutically acceptable salt, ester, acid or other variant of the defined compounds and their tautomers as well as substances which, upon administration, are capable of providing directly or indirectly a compound

### Method

In an aspect of the invention there is provided a method of synthesis of 2,2-disubstituted 3-oxindole compounds, such as those of formula (I) and (3).

Typically the compounds of formula (I) are synthesized by a cascade process wherein an a benzyne precursors is used to generate benzyne, the benzyne obtained captures the amine (proline derivative), this is followed by a cyclisation, an ylide (neutral dipolar molecule containing a negatively charged carbon atom directly bonded to a positively charged heteroatom (usually nitrogen, phosphorus or sulfur)) formation, which undergoes a [2,3]-sigmatropic rearrangement, derivative, thereby generating 3-oxindoles including a quaternary centre.

For synthesis of the compounds of formula (3) the amine is a malonate derivative.

The method of synthesis generally produces compounds of formula (1) and follows reaction scheme (1).

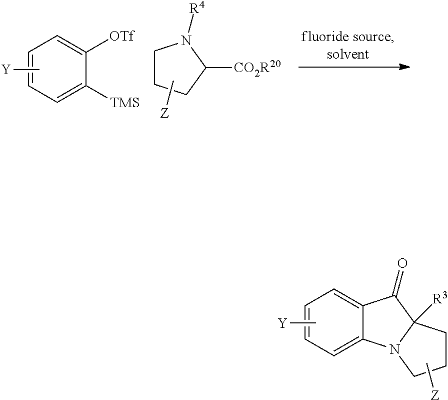

Y, Z, X, R1, R2, R15, R16 and X are as defined above. R4 represents an alkenyl group, or an alkynyl group.

Typically R4 represents a 2-alkenyl group or a 2-alkynyl group.

Where OTf is used to represent trifluoromethane sulfonate and TMS is used to represent trimethylsilyl.

Suitable fluoride sources include CsF, TBAF, TMAF, TBAT and KF. Suitable solvents include acetonitrile DME, diglyme and THF. Typically, the reaction proceeds for around 2 to 3 hours at room temperature.

The reaction mixture is generally prepared under an inert atmosphere such as nitrogen.

In one embodiment, R4 is an alkynyl group, thereby forming an N-propargyl proline. In such embodiments, one of R1 and R2 is not present. Typically, the R4 alkyne tail is any one of the following N-propargyl proline family:

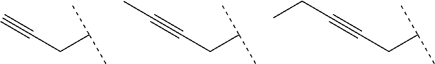

According to a further aspect of the present invention, there is provided a method of synthesis of the compounds of formula (3), whereby 2-(trimethylsilyl)phenyl trifluoromethanesulfonate or a derivative thereof and dimethylaminomalonate (suitably dimethyl 2-(allyl(methyl)amino)malonate) or a derivative thereof are mixed in the presence of a fluoride source such as CsF, TBAF, TMAF, TBAT and KF typically in a suitable solvent such as CH₃CN (acetonitrile), DME, diglyme and THF.

According to an aspect of the present invention, there is provided a method of synthesising the compounds of formula (3) according to reaction scheme A:

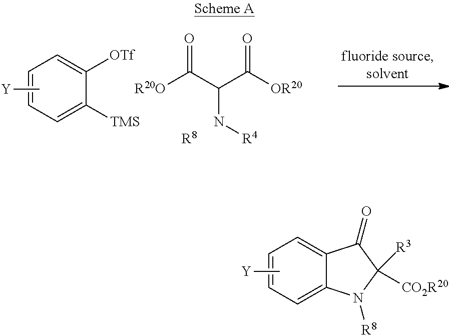

Y, R8, R20, R4 and R3 are as defined above.

Suitable fluoride sources include CsF, TBAF, TMAF, TBAT and KF. Suitable solvents include acetonitrile, DME, THF and diglyme.

Typically the reaction proceeds for around 2 to 3 hours at room temperature.

The reaction mixture is generally prepared under an inert atmosphere such as nitrogen.

Typically the method of synthesis follows the scheme C below:

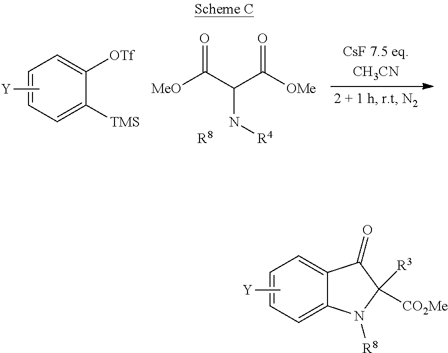

Y, R8, R4 and R3 are as defined above.

In one embodiment the fluoride source is cesium fluoride or tetrabutylammonium fluoride (TBAF).

For the reaction schemes above, the benzyne precursor:

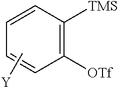

may have one of the following structures:

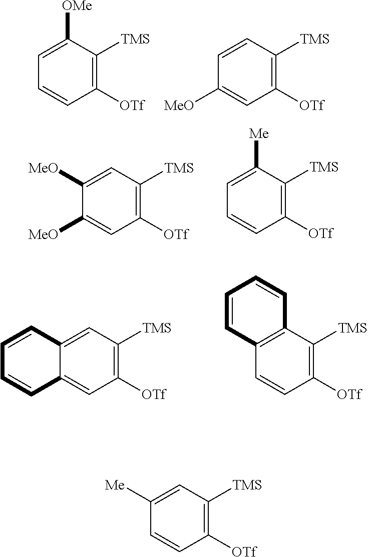

The methods of the present invention allow compounds with tailored, predictable properties to be synthesised. By choice of reagents, the methods also allow the manufacture of tuned fluorescers, with ‘dialled-in’ absorption/emission properties, to suit end-user needs.

The methods of the present invention tend to have a higher efficiency and higher associated yield than prior art methods of producing 3-oxindoles. Where the method is used to synthesise 2,2-disubstituted 3-oxindole compounds (for instance by the methods of Scheme (1)), the associated yield is generally at least 30%, typically at least 44%.

Where the method is used to synthesise di-substituted 3-oxindole compounds (for instance by the methods of Scheme A), the associated yield is generally at least 48%.

### Uses

According to an aspect of the present invention, there is provided the use of the compounds disclosed herein as fluorescent labelling agents.

The compounds of the present invention can be used, for example, to measure the amount of a molecular target drug (in particular an amine containing target drug) binding to a biological target.

For example, a drug tagged with a compound having the structure of formula (I) is provided below:

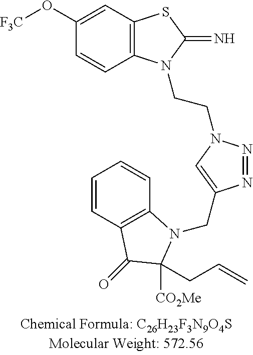 [SMILES: C=CCC1(C(=O)OC)C(=O)c2ccccc2N1Cc1cn(CCn2c(=N)sc3cc(OC(F)(F)F)ccc32)nn1]

According to an aspect of the present invention, there is provided a method of measuring the amount of binding between a molecular target drug and a biological target wherein the molecular target drug comprises an amine group, including reacting the molecular target drug with a compound as described herein, administering the resultant product to a sample comprising the biological target and monitoring fluorescence emitted from the sample.

The compounds disclosed herein may be used to image biological structures such as organelles of cancer cells.

The compounds of the present invention find utility in ex-vivo or in-vitro imaging applications.

The compounds of the present invention can be used as bioprobes in cell imaging, with high photostability and brightness. The compounds exhibit a large Stokes shift (>110 nm), high biocompatibility, and good photostability.

The compounds of the present invention have a relatively low molecular weight. Generally, the associated number average molecular weight is less than 500 g/mol.

The compounds of the present invention are generally uncharged.

According to a further aspect of the present invention, there is provided a method of tagging a compound comprising an amine group comprising contacting the compound comprising an amine group with a compound disclosed herein and monitoring fluorescence emitted.

Where the compounds disclosed herein are used to stain living tissue, the compounds are generally provided at concentrations of 1 to 100 μm.

The present invention will now be described by way of Example only with reference to the accompanying figures.

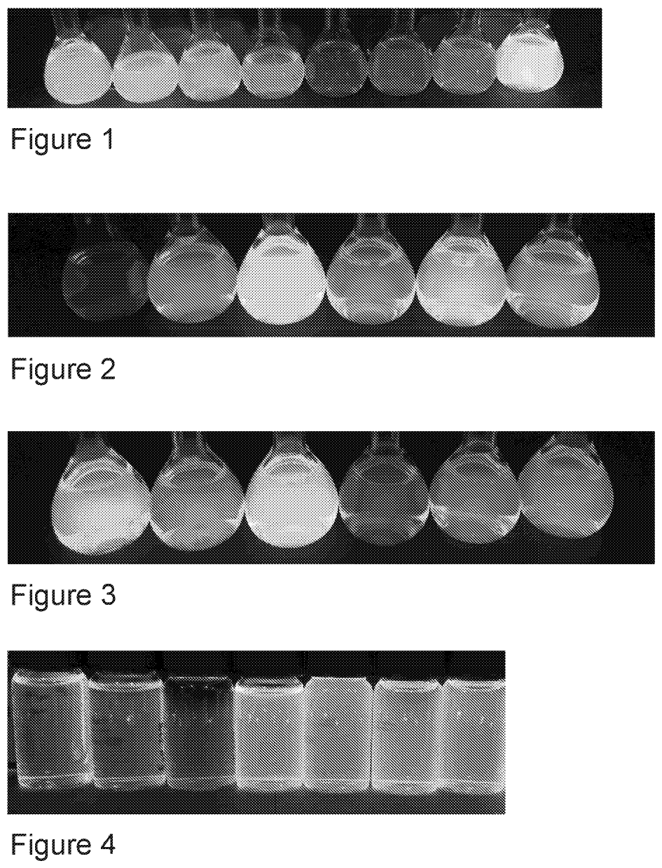

FIG. 1 is a photo demonstrating the fluorescence of the compounds of the present invention. From left to right compounds 3ab, 3aa, 3bb, 3db, 3bc, 3eb, 3eb′ in DCM illuminated at 365 nm (structures provided below).

FIG. 2 is a photo demonstrating the fluorescence of the compounds of the present invention. From left to right compounds 3bh, 3cb, 3hb, 3ba, 3da, 3ca in DCM illuminated at 365 nm (structures provided below).

FIG. 3 is a photo demonstrating the fluorescence of the compounds of the present invention. From left to right compounds 3fa, 3fa′, 3ea, 3ea′, 3bd, 3ae in DCM illuminated at 365 nm (structures provided below).

FIG. 4 is a photo demonstrating the fluorescence of the compounds of the present invention. From left to right compounds 99a, 99c, 99d, 99b, 99e, 99f, 99g, in DCM illuminated at 365 nm (structures provided below).

### EXAMPLES

### General Procedure for Oxindole Synthesis

### Proline Series: General Procedure 1

To a flame dried round-bottom flask, amine (5.0 eq) was added to a stirred solution of aryne precursor (1 eq) in MeCN (10 mL/mmol of aryne precursor, 100 mM) under an atmosphere of N2 at 25° C. To the mixture, tetrabutylammonium fluoride in THF (1M, 3 eq) was added over 2 h (dropwise). The mixture was run through a silica plug washing with EtOAc until all colour was removed. The filtrate was evaporated in vacuo to give the crude material which is purified by column chromatography (solutions of petrol and EtOAc as the eluent).

### Malonate Series: General Procedure 2

CsF (7.5 eq) was added to a round bottom flask, and the flask was flame dried under vacuum. The flask was then filled with N2, a stirrer bar was added and the flask was briefly heated under high vacuum (the stirrer bar could melt if added at the same time as CsF) before being filled with N2. Then, the amine (1 eq) and MeCN (45 mL/mmol amine, 22 mM) were added and stirred under an atmosphere of N2 at 25° C. Then a solution of aryne precursor (1.5 eq) in MeCN (1 M) was added dropwise using a syringe pump over two hours, and the reaction mixture was left to stir 1 hour after that. The reaction mixture was then filtered through a celite plug, and the celite was washed with EtOAc (until the bright yellow colour was removed). The filtrate was concentrated under reduced pressure and the residue was purified by silica gel column chromatography.

### Malonate Series: General Procedure 3

To a flame dried round-bottom flask, amine (1.5 eq) was added to a stirred solution of aryne precursor (1 eq) in MeCN (10 mL/mmol of aryne precursor, 100 mM) under an atmosphere of N2 at 25° C. To the mixture tetrabutylammonium fluoride solution (1 M, 2.9 eq) was added over 2 h (dropwise). The mixture was run through a silica plug washing with EtOAc until all colour is removed. The filtrate was evaporated in vacuo to give the crude material which is purified by column chromatography (solutions of petrol and EtOAc or Et2O as the eluent).

### 9a-allyl-1,2,3,9a-tetrahydro-9H-pyrrolo[1,2-a]indol-9-one 99a

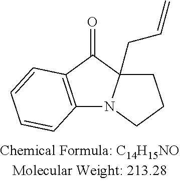 [SMILES: C=CCC12CCCN1c1ccccc1C2=O]

To a flame dried round-bottom flask flushed with nitrogen, methyl allyl-L-prolinate (23, 280 mg, 1.65 mmol, 5 eq) was added to solution of 2-(trimethylsilyl)phenyl trifluoromethanesulfonate (80 IL, 0.33 mmol, 1 eq) in acetonitrile (10 mL) stirring at r.t. under nitrogen. Dried TBAF (1 M in THF, 1.32 mL, 1.32 mmol, 4.0 eq) was then added to the resulting solution, over 2 h (dropwise, using a syringe pump). The mixture was filtered and eluted through a silica plug washing with EtOAc first until all colour was removed (˜100 mL), and then with more EtOAc (100 mL) in order to get all the leftover methyl allyl-L-prolinate. The filtrate was evaporated in vacuo to give the crude material which was then purified by column chromatography (50 g silica, Hexane:EtOAc, 10:1) affording the title compound as a fluorescent yellow oil (31 mg, 44%).

¹H NMR (CDCl₃, 400 MHz) δ_{H} 7.55 (d, J7.7 Hz, 1H), 7.49-7.53 (m, 1H), 6.94 (d, J 8.2 Hz, 1H), 6.91 (t, J 7.4 Hz, 1H), 5.67-5.77 (m, 1H), 5.13 (dm, J 17.1 Hz, 1H), 5.02 (dm, J 10.2 Hz, 1H), 3.47-3.52 (m, 1H), 3.32-3.37 (m, 1H), 2.58 (dd, J 7.3 Hz, 13.9 Hz, 1H), 2.48 (dd, J 7.0 Hz, 13.9 Hz, 1H), 2.00-2.07 (m, 1H), 1.77-1.92 (m, 3H).

¹³C NMR (CDCl_{3, 100} MHz) δc 206.2 (1C); 165.2 (1C), 137.1 (1CH), 132.9 (1CH), 124.4 (1CH), 123.8 (1C), 120.5 (1CH), 118.6 (1CH₂), 114.2 (1CH), 78.2 (1C), 51.2 (1CH₂), 40.6 (1CH₂), 31.8 (1CH₂), 26.9 (1CH₂).

IR υmax (thin film, cm⁻¹) 3074, 2967, 2882, 1698, 1606, 1474.

UV-Vis (EtOH) λmax (nm), Σ(M⁻¹ cm⁻¹) 213 (5729), 230 (6388), 327 (1103), 388 (2462).

HRMS m/z (ESI⁺) calculated for C₁₄H₁₅NO [M+H]+214.1226, found 214.1228 (error −0.87 ppm).

### 9a-(2-Methylallyl)-2,3-dihydro-1H-pyrrolo[1,2-a]indo1-9(9aH)-one 99b

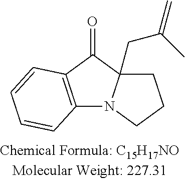 [SMILES: C=C(C)CC12CCCN1c1ccccc1C2=O]

According to general procedure 3, (S)-methyl 1-(2-methylallyl)pyrrolidine-2-carboxylate was reacted with 2-(trimethylsilyl)phenyl trifluoromethanesulfonate to yield the title compound (15%) after column chromatography (9.5:0.5 v/v petrol:EtOAc) as fluorescent yellow oil. Rf=0.25 (9.5:0.5 v/v petrol:EtOAc)

¹H NMR (CDCl₃, 400 MHz) δH 1.68 (s, 3H), 1.72-1.77 (m, 1H), 1.84-1.90 (m, 1H), 1.92-1.97 (m, 1H), 2.05-2.12 (m, 1H), 2.42 (d, J 13.7 Hz, 1H), 2.66 (d, J 13.7 Hz, 1H), 3.34 (dd, J 6.6 Hz 17.9 Hz, 1H), 3.51 (dd, J 7.0 Hz 17.9 Hz, 1H), 4.73 (m, 2H), 6.90 (t, J 7.8 Hz, 1H), 6.91 (d, J 8.5 Hz, 1H), 7.50 (td, J 1.3 Hz, 7.0 Hz, 1H), 7.55 (d, J 7.6 Hz, 1H).
¹³C NMR (CDCl₃, 100 MHz) δC 24.0 (1CH₃), 27.0 (1CH₂), 32.7 (1CH₂), 43.5 (1CH₂), 50.7 (1CH₂), 78.6 (1C), 114.0 (1CH), 114.3 (1CH₂) 120.3 (1CH), 123.6 (1C), 124.5 (1CH), 137.0 (1CH), 141.6 (1C), 165.1 (1C), 206.0 (1C).

IR υₘₐₓ (thin film, cm⁻¹): 1702 (C═O aryl), 1316 (C—N aryl).

UV-Vis (EtOH)λmax (nm), Σ(M⁻¹cm⁻¹) 200 (10588), 203 (9989), 236 (10856), 322 (513), 389 (999)

HRMS m/z (ESI+) calculated for C15H17NO [M+H]+; 228.1383, found 228.1386.

### 9a-(But-3-en-2-yl)-2,3-dihydro-1H-pyrrolo[1,2-a]indol-9(9aH)-one 99c

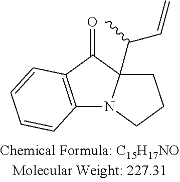 [SMILES: C=CC(C)C12CCCN1c1ccccc1C2=O]

According to general procedure 3, (S)-(E)-methyl 1-(but-2-en-1-yl)pyrrolidine-2-carboxylate was reacted with 2-(trimethylsilyl)phenyl trifluoromethanesulfonate to yield the title compound (30%, d.r. 1:0.2) after column chromatography (9.8:0.2 v/v petrol:EtOAc) as fluorescent yellow oil. Rf=0.07 (9.8:0.2 v/v petrol:EtOAc)

¹H NMR (CDCl₃, 400 MHz) δH 0.93 (d, J 6.8 Hz, 2.28H, major diastereomer), 1.05 (d, J 6.9 Hz, 0.72H, minor diastereomer), 1.66-2.09 (m, 4H), 2.51-2.59 (m, 0.22H), 2.64 (dq, J 6.7 Hz 9.0 Hz, 0.78H), 3.29-3.60 (m, 2H), 5.02 (dd, J 1.9 Hz, 10.3 Hz, 0.22H), 5.04-5.09 (m, 0.22H), 5.10 (dd, J 1.8 Hz 10.2 Hz, 0.78H), 5.16 (dd, J 1.8 Hz, 17.2 Hz, 0.78H), 5.83 (ddd, J 9.2 Hz, 10.2 Hz 17.2 Hz, 0.78H), 6.04 (ddd, J 8.8 Hz, 10.2 Hz, 17.2 Hz, 0.22H), 6.54 (d, J 7.9 Hz, 0.22H), 6.71 (t, J 7.3 Hz, 0.22H), 6.88-7.00 (m, 1.56H), 7.20-7.24 (m, 0.44H), 7.48-7.54 (m, 1.56H)

¹³C NMR (CDCl₃, 100 MHz) δC (only major diastereoisomer reported) 15.1 (1CH₃), 26.2 (1CH₂), 31.0 (1CH₂), 45.3 (1CH), 52.7 (1CH₂), 80.8 (1C), 114.7 (1CH), 116.5 (1CH₂), 120.6 (1CH), 124.0 (1CH), 125.0 (1C), 137.0 (1CH), 140.0 (1CH), 166.3 (1C), 207.8 (1C).

IR υₘₐₓ (thin film, cm⁻¹): 1697 (C═O aryl), 1310 (C—N aryl).

UV-Vis (EtOH) λmax (nm), Σ(M⁻¹cm⁻¹) 202 (8636), 206 (8610), 235 (14460), 327 (799), 389 (1295)

HRMS m/z (ESI+) calculated for C15H17NO [M+H]+; 228.1383, found 228.1385.

### 9a-(2-Methylbut-3-en-2-yl)-2,3-dihydro-1H-pyrrolo[1,2-a]indol-9(9aH)-one99d

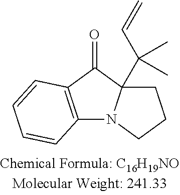 [SMILES: C=CC(C)(C)C12CCCN1c1ccccc1C2=O]

According to general procedure 3, (S)-methyl 1-(3-methylbut-2-en-1-yl)pyrrolidine-2-carboxylate was reacted with 2-(trimethylsilyl)phenyl trifluoromethanesulfonate to yield the title compound (50%) after column chromatography (9.8:0.2 v/v petrol:EtOAc) as fluorescent yellow oil. Rf=0.08 (9.8:0.2 v/v petrol:EtOAc)

¹H NMR (CDCl₃, 400 MHz) δH 1.05 (s, 3H), 1.14 (s, 3H), 1.74 (d, J 15.9 Hz, 2H), 1.94-1.99 (m, 2H), 3.23 (td, J 5.4 Hz 14.0 Hz, 1H), 3.42 (td, J 7.2 Hz 14.0 Hz, 1H), 5.03 (br s, 1H), 5.07 (dd, J 1.4 Hz 7.4 Hz, 1H), 6.11 (dd, J 11.1 Hz 17.2 Hz, 1H), 6.91 (t, J 7.4 Hz, 1H), 6.98 (d, J 8.4 Hz, 1H), 7.49 (t, J 7.1 Hz, 1H), 7.49 (dd, J 1.3 Hz 7.6 Hz, 1H).

¹³C NMR (CDCl₃, 100 MHz) δC 21.1 (1CH₃), 22.5 (1CH₃), 25.7 (1CH₂), 29.6 (1CH₂), 43.6 (1C), 53.7 (1CH₂), 82.5 (1C), 113.2 (1CH₂), 115.0 (1CH), 120.8 (1CH), 123.6 (1CH), 126.4 (1C), 136.7 (1CH), 144.5 (1CH), 165.9 (1C), 208.5 (1C).

IR υₘₐₓ (thin film, cm⁻¹): 1699 (C═O aryl), 1311 (C—N aryl).

UV-Vis (EtOH) λmax (nm), Σ(M⁻¹cm⁻¹) 203 (6629), 236 (6705), 320 (496), 386 (483).

HRMS m/z (ESI+) calculated for C16H19NO [M+H]+; 242.1539, found 242.1545.

### 9a-(Propa-1,2-dien-1-yl)-2,3-dihydro-1H-pyrrolo[1,2-a]indol-9(9aH)-one 99e

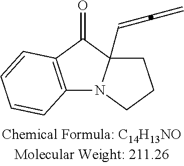

According to general procedure 3, (S)-methyl 1-(prop-2-yn-1-yl)pyrrolidine-2-carboxylate was reacted with 2-(trimethylsilyl)phenyl trifluoromethanesulfonate to yield the title compound (60%) after column chromatography (9.5:0.5 v/v petrol:EtOAc) as fluorescent yellow oil.

Rf=0.22 (9.5:0.5 v/v petrol:EtOAc).

¹H NMR (CDCl₃, 400 MHz) δH 1.70 (dd, J 7.5 Hz 12.2 Hz, 1H), 2.04-2.17 (m, 2H), 2.19-2.28 (m, 1H), 3.24 (dt, J 7.5 Hz 10.4 Hz, 1H), 3.61 (ddd, J 4.1 Hz 7.9 Hz 10.2 Hz, 1H), 4.92 (dd, J 6.5 Hz 11.0 Hz, 1H), 4.98 (dd, J 6.6 Hz 11.0 Hz, 1H), 5.33 (t, J 6.6 Hz, 1H), 6.89 (d, J 8.2 Hz, 1H), 6.91 (t, J 7.5 Hz, 1H), 7.52 (t, J 7.7 Hz, 1H), 7.58 (d, J 7.7 Hz, 1H).

¹³C NMR (CDC₃, 100 MHz) δC 27.7 (1CH₂), 32.0 (1CH₂), 50.5 (1CH₂), 77.2 (1C), 78.5 (1CH₂), 92.1 (1CH), 113.8 (1CH), 120.4 (1CH), 122.4 (1C), 125.0 (1CH), 137.4 (1CH), 164.6 (1C), 202.9 (1C), 207.8 (1C).

IR υₘₐₓ (thin film, cm⁻¹): 1701 (C═O aryl), 1316 (C—N aryl).

UV-Vis (EtoH) λmax (nm), Σ(M⁻¹cm⁻¹) 234 (13006), 335 (1068), 391 (2404).

HRMS m/z (ESI+) calculated C14H13NO [M+H]+; 212.1070, found 212.1081.

9a-(Buta-2,3-dien-2-yl)-2,3-dihydro-1H-pyrrolo[1,2-a]indol-9(9aH)-one 99f

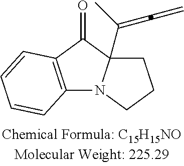

According to general procedure 3, (S)-methyl 1-(but-2-yn-1-yl)pyrrolidine-2-carboxylate was reacted with 2-(trimethylsilyl)phenyl trifluoromethanesulfonate to yield the title compound (50%) after column chromatography (9.5:0.5 v/v petrol:EtOAc) as fluorescent yellow oil which solidified on standing to a waxy fluorescent yellow solid.

Rf=0.28 (9.5:0.5 v/v petrol:EtOAc).

mp: 73-74° C.

¹H NMR (CDCl₃, 400 MHz) δH 1.59-1.64 (m, 1H), 1.62 (t, J 3.1 Hz, 3H), 1.99-2.06 (m, 1H), 2.11-2.18 (m, 1H), 2.28-2.34 (m, 1H), 3.26 (dt, J 7.5 Hz 10.5 Hz, 1H), 3.54 (ddd, J 4.9 Hz, 7.9 Hz, 10.6 Hz, 1H), 4.85 (dq, J 3.1 Hz 10.1 Hz, 1H), 4.95 (dq, J 3.1 Hz, 10.2 Hz, 1H), 6.90 (t, J 7.2 Hz, 1H), 6.91 (d, J 8.5 Hz, 1H), 7.52 (ddd, J 1.0 Hz, 7.2 Hz, 8.2 Hz, 1H), 7.57 (d, J 7.7 Hz, 1H).

¹³C NMR (CDCl₃, 100 MHz) ^{TM}C 14.5 (1CH₃), 27.9 (1CH₂), 30.5 (1CH₂), 50.1 (1CH₂), 77.5 (1CH₂), 79.3 (1C), 99.2 (1C), 113.7 (1CH), 120.2 (1CH), 123.0 (1C), 124.7 (1CH), 137.3 (1CH), 165.3 (1C), 204.0 (1C), 206.5 (1C).

IR υₘₐₓ (thin film, cm⁻¹): 1704 (C═O aryl), 1320 (C—N aryl).

UV-Vis (EtOH) λmax (nm), Σ(M⁻¹cm⁻¹) 233 (12934), 337 (1092), 392 (2350)

HRMS m/z (ESI+) calculated for C15H15NO [M+H]+; 226.1226, found 226.1225.

### 9a-(Penta-1,2-dien-3-yl)-2,3-dihydro-1H-pyrrolo[1,2-a]indol-9(9aH)-one 99g

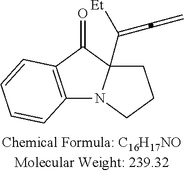

According to general procedure 3, (S)-methyl 1-(pent-2-yn-1-yl)pyrrolidine-2-carboxylate was reacted with 2-(trimethylsilyl)phenyl trifluoromethanesulfonate to yield the title compound (50%) after column chromatography (9.5:0.5 v/v petrol:EtOAc) as fluorescent yellow oil.

Rf=0.35 (9.5:0.5 v/v petrol:EtOAc).

¹H NMR (CDCl₃, 400 MHz) δH 0.95 (t, J 7.3 Hz, 3H), 1.55-1.63 (m, 1H), 1.71-1.82 (m, 1H), 1.93-2.09 (m, 2H), 2.11-2.20 (m, 1H), 2.35 (ddd, J 3.9 Hz, 6.7 Hz, 12.5 Hz, 1H), 3.26 (dt, J 7.6 Hz 10.5 Hz, 1H), 3.54 (ddd, J 4.7 Hz 8.0 Hz, 10.6 Hz, 1H), 4.97 (dt, J 3.8 Hz 9.9 Hz, 1H), 5.08 (dt, J 3.8 Hz 9.9 Hz, 1H), 6.90 (d, J 8.2 Hz, 1H), 6.90 (t, J 7.3 Hz, 1H), 7.51 (t, J 7.6 Hz, 1H), 7.56 (d, J 7.7 Hz, 1H).

¹³C NMR (CDCl₃, 100 MHz) δC 12.2 (1CH₃), 20.1 (1CH₂), 28.0 (1CH₂), 30.8 (1CH₂), 50.1 (1CH₂), 79.4 (1C), 80.0 (1CH₂), 106.3 (1C), 113.6 (1CH), 120.2 (1CH), 123.0 (1C), 124.8 (1CH), 137.2 (1CH), 165.3 (1C), 204.2 (1C), 205.8 (1C).

IR υₘₐₓ (thin film, cm⁻¹): 1705 (C═O aryl), 1319 (C—N aryl).

UV-Vis (EtOH) δmax (nm), Σ(M⁻¹cm⁻¹) 228 (13711), 232 (33376), 236 (33376), 337 (3189), 391 (6833).

HRMS m/z (ESI+) calculated for C16H17NO [M+H]+; 240.1383, found 240.1381.

### Methyl 2-allyl-1-methyl-3-oxoindoline-2-carboxylate 3aa

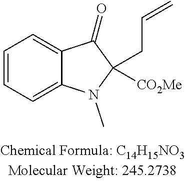 [SMILES: C=CCC1(C(=O)OC)C(=O)c2ccccc2N1C]

According to general procedure 2, dimethyl 2-(allyl(methyl)amino)malonate (133 mg, 0.67 mmol, 1 eq) was reacted with 2-(trimethylsilyl)phenyl trifluoromethanesulfonate (297 mg, 1 mmol, 1.5 eq) to yield compound 3aa (99 mg, 61%) as a bright yellow solid after silica gel column chromatography (Toluene:EtOAc 20:1)

mp 90-93° C.

¹H NMR (CDCl₃, 400 MHz) δ_{H} 7.55 (d,J 7.7 Hz, 1H), 7.46-7.50 (m, 1H), 6.79 (d,J 8.4 Hz, 1H), 6.73 (app. t, J 7.4 Hz, 1H), 5.34-5.45 (m, 1H), 5.14 (app dm, J 16.9 Hz, 1H), 4.97 (app dm, J 10.2 Hz, 1H), 3.70 (s, 3H), 3.06 (dd, J 6.8 Hz, 14.6 Hz, 1H), 2.99 (s, 3H), 2.89 (dd, J 7.5 Hz, 14.5 Hz, 1H).

¹³C NMR (CDCl₃, 100 MHz) δc 195.3 (1C), 167.6 (1C), 161.7 (1C), 138.0 (1CH), 131.6 (1CH), 125.1 (1CH), 119.7 (1CH₂), 119.0 (1C), 117.6 (1CH), 108.3 (1CH), 76.8 (1C), 53.0 (1CH₃), 36.5 (1CH₂), 29.1 (1CH₃).

IR υₘₐₓ (thin film, cm⁻¹): 3074, 2952, 2917, 2848, 1739, 1696, 1612, 1489.

UV-Vis (EtOH) λₘₐₓ (nm), Σ(M⁻¹ cm⁻¹) 231 (8707), 416 (5386).

HRMS m/z (ESI⁺) calculated for C₁₄H₁₅NO₃[M+H]·: 246.1125, found 246.1131.

### Methyl 2-allyl-4-methoxy-1-methyl-3-oxoindoline-2-carboxylate 3ba

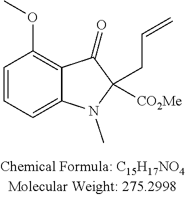 [SMILES: C=CCC1(C(=O)OC)C(=O)c2c(OC)cccc2N1C]

According to general procedure 2, dimethyl 2-(allyl(methyl)amino)malonate (133 mg, 0.67 mmol, 1 eq) was reacted with 3-methoxy-2-(trimethylsilyl)phenyl trifluoromethanesulfonate (327 mg, 1.0 mmol, 1.5 eq) to yield compound 3ba (116 mg, 64%) as a bright yellow solid after silica gel column chromatography (petrol:acetone, 3:1).

mp: 125-127° C.

¹H NMR (CDCl₃, 400 MHz) δH 7.39 (app. t, J 8.2 Hz, 1H), 6.33 (d, J 8.2 Hz, 1H), 6.14 (d, J 8.1 Hz, 1H), 5.39-5.49 (m, 1H), 5.14 (ddd, J 1.4 Hz, 3.0 Hz, 17.0 Hz, 1H), 4.98 (app. dm, J 10.1 Hz, 1H) 3.89 (s, 3H), 3.69 (s, 3H), 3.07 (app. ddt, J 1.1 Hz, 6.7 Hz, 14.6 Hz, 1H), 2.96 (s, 3H), 2.85 (app. ddt, J 1.1 Hz, 7.6 Hz, 14.6 Hz, 1H).

¹³C NMR (CDCl₃, 100 MHz) 6_{c} 192.4 (1C), 167.7 (1C), 163.0 (1C), 159.2 (1C), 139.6 (1CH), 130.8 (1CH), 119.6 (1CH₂), 108.1 (1C), 100.5 (1CH), 98.9 (1CH), 77.0 (1C), 55.8 (1CH₃), 53.0 (1CH₃), 36.5 (1CH₂), 29.3 (1CH₃).

IR υₘₐₓ (thin film, cm⁻¹): 2920, 1738, 1688, 1603, 1583, 1501.

HRMS m/z (ESI+) calculated for C₁₅H₁₇NO₄[M+H]⁺; 276.123, observed 276.1226.

### Methyl 2-allyl-1,4-dimethyl-3-oxoindoline-2-carboxylate 3ca

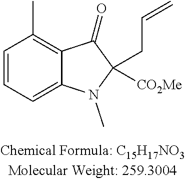 [SMILES: C=CCC1(C(=O)OC)C(=O)c2c(C)cccc2N1C]

According to general procedure 2, dimethyl 2-(allyl(methyl)amino)malonate (133 mg, 0.67 mmol, 1 eq) was reacted with 3-methyl-2-(trimethylsilyl)phenyl trifluoromethanesulfonate (311 mg, 1 mmol, 1.5 eq) to yield compound 3ca (92 mg, 54%) as a bright yellow solid after silica gel column chromatography (Petrol:Et₂O 8:1).

mp 98-100° C.

¹H NMR (CDCl₃, 400 MHz) δ_{H} 7.33 (app. t, J 7.8 Hz, 1H), 6.59 (d, J 8.3 Hz, 1H), 6.49 (d, J 7.2 Hz, 1H), 5.36-5.47 (m, 1H), 5.14 (d, J 16.9 Hz, 1H), 4.97 (d, J 10.1 Hz, 1H), 3.71 (s, 3H), 3.05 (dd, J 6.7 Hz, 14.6 Hz, 1H), 2.96 (s, 3H), 2.87 (dd, J 7.6 Hz, 14.6 Hz, 1H), 2.53 (s, 3H).

¹³C NMR (CDCl₃, 100 MHz) 6_{c} 195.5 (1C), 167.9 (1C), 162.3 (1C), 140.6 (1C), 137.2 (1CH), 130.9 (1CH), 119.4 (1CH₂), 119.2 (1CH), 117.3 (1C), 105.4 (1CH), 76.7 (1C), 53.0 (1CH₃), 36.7 (1CH₂), 29.2 (1CH₃), 18.3 (1CH₃).

IR υₘₐₓ (thin film, cm⁻¹): 2952, 2920, 1735, 1678, 1601, 1496.

HRMS m/z (ESI+) calculated for C₁₅H₁₇NO₃[M+H]⁺; 260.1281, observed 260.1278.

### Methyl 2-allyl-3-methyl-1-oxo-2,3-dihydro-1H-benzo[e]indole-2-carboxylate 3da

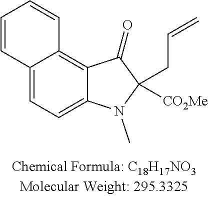 [SMILES: C=CCC1(C(=O)OC)C(=O)c2c(ccc3ccccc23)N1C]

According to general procedure 2, dimethyl 2-(allyl(methyl)amino)malonate (133 mg, 0.67 mmol, 1 eq) was reacted with 1-(trimethylsilyl)naphthalen-2-yl trifluoromethanesulfonate (347 mg, 1 mmol, 1.5 eq) to yield compound 3da (131 mg, 67%) as a bright yellow solid after silica gel column chromatography (Petrol : EtOAc, 8:1).

### mp: 144-147° C.

¹H NMR (CDCl₃, 400 MHz) δH 8.70 (d, J 8.3 Hz, 1H), 7.94 (d, J 9.0 Hz, 1H), 7.71 (d, J 8.0 Hz, 1H), 7.58 (ddd, J 1.2 Hz, 7.1 Hz, 8.3 Hz, 1H), 7.31 (ddd, J 1.1 Hz, 7.1 Hz, 8.2 Hz, 1H), 7.06 (d, J 9.0 Hz, 1H), 5.39-5.49 (m, 1H), 5.17 (ddd, J 1.4 Hz, 2.9 Hz, 16.9 Hz, 1H), 4.95 (app. dm, J 10.1 Hz, 1H), 3.73 (s, 3H), 3.20 (app. ddt, J 1.2 Hz, 6.3 Hz, 14.6 Hz, 1H), 3.13 (s, 3H), 2.93 (dd, J 7.9 Hz, 16.6 Hz, 1H).

¹³C NMR (CDCl₃, 100 MHz) δ_{c} 193.1 (1C), 167.5 (1C), 164.1 (1C), 140.0 (1CH), 130.8 (1CH), 130.5 (1C), 130.1 (1CH), 128.5 (1CH), 127.1 (1C), 123.6 (1CH), 122.7 (1CH), 119.5 (1CH₂), 110.0 (1CH), 109.3 (1C), 77.6 (1C), 53.1 (1CH₃), 36.8 (1CH₂), 29.4 (1CH₃).

IR υₘₐₓ (thin film, cm⁻¹): 1737, 1657, 1625, 1588, 1567, 1532.

HRMS m/z (ESI+) calculated for C₁₈H₁₇NO₃[M+H]⁺; 296.1281, observed 296.1278.

### Methyl 2-allyl-5-methoxy-1-methyl-3-oxoindoline-2-carboxylate 3ea

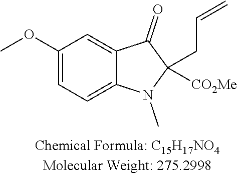 [SMILES: C=CCC1(C(=O)OC)C(=O)c2cc(OC)ccc2N1C]

According to general procedure 2, dimethyl 2-(allyl(methyl)amino)malonate (133 mg, 0.67 mmol, 1 eq) was reacted with 4-methoxy-2-(trimethylsilyl)phenyl trifluoromethanesulfonate (327 mg, 1 mmol, 1.5 eq) to yield compounds 3ea (67 mg, 37%) as a bright yellow oil and 3ea′ (55 mg, 30%) as a bright yellow solid, after silica gel column chromatography (Petrol : EtOAc, 9:1).

¹H NMR (CDCl₃, 400 MHz) δH 7.18 (dd, J 2.6 Hz, 8.9 Hz, 1H), 7.01 (d, J 2.6 Hz, 1H), 6.76 (d,J 8.9 Hz, 1H), 5.36-5.44 (m, 1H), 5.14 (d, J 16.8 Hz, 1H), 4.97 (d, J 10.1 Hz, 1H), 3.75 (s, 3H), 3.71 (s, 3H), 3.04 (dd, J 6.8 Hz, 14.6 Hz, 1H), 2.97 (s, 3H), 2.88 (dd, J 7.4 Hz, 14.6 Hz, 1H).

¹³C NMR (CDCl₃, 100 MHz) δC 195.2 (1C), 167.8 (1C), 158.1 (1C), 152.4 (1C), 130.8 (1CH), 128.7 (1CH), 119.6 (1CH₂), 118.9 (1C), 109.7 (1CH), 105.0 (1CH), 77.5 (1C), 55.8 (1CH₃), 53.0 (1CH₃), 36.5 (1CH₂), 29.4 (1CH₃).

IR υₘₐₓ (thin film, cm⁻¹): 1735, 1678.

HRMS m/z (ESI+) calculated for C₁₅H₁₇NO₄[M+H]⁺; 276.1230, observed 276.1227.

Methyl 2-allyl-6-methoxy-1-methyl-3-oxoindoline-2-carboxylate 3ea′

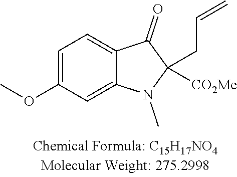 [SMILES: C=CCC1(C(=O)OC)C(=O)c2ccc(OC)cc2N1C]

According to general procedure 2, dimethyl 2-(allyl(methyl)amino)malonate (133 mg, 0.67 mmol, 1 eq) was reacted with 4-methoxy-2-(trimethylsilyl)phenyl trifluoromethanesulfonate (327 mg, 1 mmol, 1.5 eq) to yield compounds 3ea (67 mg, 37%) as a bright yellow oil and 3ea′ (55 mg, 30%) as a bright yellow solid, after silica gel column chromatography (Petrol : EtOAc, 9:1).

mp 99-101° C.

¹H NMR (CDCl₃, 300 MHz) δ_{H} 7.49 (d, J 8.6 Hz, 1H), 6.33 (ddd, J 0.8 Hz, 2.1 Hz, 8.6 Hz, 1H), 6.15 (d, J 1.9 Hz, 1H), 5.35-5.49 (m, 1H), 5.14 (app. dm, 17.0 Hz, 1H), 4.98 (app. dm, J 10.0 Hz, 1H), 3.89 (s, 3H), 3.72 (s, 3H), 3.08 (dd, J 6.6 Hz, 14.6 Hz, 1H), 2.98 (s, 3H), 2.85 (dd, J 7.6 Hz, 14.6 Hz, 1H).

¹³C NMR (CDCl₃, 100 MHz) δ_{c} 192.5 (1C), 168.5 (1C), 167.9 (1C), 163.8 (1C), 130.8 (1CH), 126.7 (1CH), 119.5 (1CH₂), 112.7 (1C), 107.2 (1CH), 91.0 (1CH), 77.5 (1C), 55.6 (1CH₃), 53.0 (1CH₃), 36.5 (1CH₂), 29.2 (1CH₃).

IR υₘₐₓ (thin film, cm⁻¹): 1734, 1670.

HRMS m/z (ESI+) calculated for C₁₅H₁₇NO₄[M+H]⁺; 276.1230, observed 276.1229.

### Methyl 2-allyl-1,5-dimethyl-3-oxoindoline-2-carboxylate 3fa

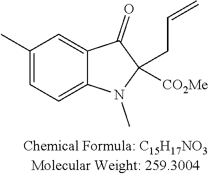 [SMILES: C=CCC1(C(=O)OC)C(=O)c2cc(C)ccc2N1C]

According to general procedure 2, dimethyl 2-(allyl(methyl)amino)malonate (133 mg, 0.67 mmol, 1 eq) was reacted with 4-methyl-2-(trimethylsilyl)phenyl trifluoromethanesulfonate (310 mg, 1 mmol, 1.5 eq) to yield compounds 3fa (54 mg, 31%) as a bright yellow solid and 3fa′ (57 mg, 33%) as a bright yellow solid, after silica gel column chromatography (Petrol:Et₂O, 6:1). mp 75-77° C.

¹H NMR (CDCl₃, 400 MHz) δ_{H} 7.35 (br. s, 1H), 7.32 (d, J 8.4 Hz, 1H), 6.71 (d, J 8.4 Hz, 1H), 5.35-5.45 (m, 1H), 5.13 (app. d, J 17.0 Hz, 1H), 4.96 (d, J 10.1 Hz, 1H), 3.70 (s, 3H), 3.05 (dd, J 6.8 Hz, 14.6 Hz, 1H), 2.97 (s, 3H), 2.87 (dd, J 7.5 Hz, 14.6 Hz, 1H), 2.26 (s, 3H).

¹³C NMR (CDCl₃, 100 MHz) δ_{C} 195.3 (1C), 167.8 (1C), 160.4 (1C), 139.5 (1CH), 130.8 (1CH), 127.1 (1C), 124.5 (1CH), 119.6 (1CH₂), 119.1 (1C), 108.2 (1CH), 77.1 (1C), 53.0 (1CH₃), 36.4 (1CH₂), 29.2 (1CH₃), 20.3 (1CH₃).

IR υₘₐₓ (thin film, cm⁻¹): 2952, 2920, 1737, 1686, 1620, 1575, 1504.

HRMS m/z (ESI+) calculated for C₁₅H₁₇NO₃[M+H]⁺; 260.1279, observed 260.1281.

Methyl 2-allyl-1,6-dimethyl-3-oxoindoline-2-carboxylate 3fa′

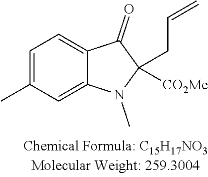 [SMILES: C=CCC1(C(=O)OC)C(=O)c2ccc(C)cc2N1C]

According to general procedure 2, dimethyl 2-(allyl(methyl)amino)malonate (133 mg, 0.67 mmol, 1 eq) was reacted with 4-methyl-2-(trimethylsilyl)phenyl trifluoromethanesulfonate (310 mg, 1 mmol, 1.5 eq) to yield compounds 3fa (54 mg, 31%) as a bright yellow solid and 3fa′ (57 mg, 33%) as a bright yellow solid, after silica gel column chromatography (Petrol:Et₂O, 6:1).

mp 89-91° C.

¹H NMR (CDCl₃, 400 MHz) δ_{H} 7.44 (d, J 7.9 Hz, 1H), 6.58 (br. s, 1H), 6.56 (d, J 8.1 Hz, 1H), 5.34-5.44 (m, 1H), 5.13 (d, J 17.0 Hz, 1H), 4.96 (d,J 10.1 Hz, 1H), 3.69 (s, 3H), 3.05 (dd, J 6.7 Hz, 14.6 Hz, 1H), 2.97 (s, 3H), 2.86 (dd, J 7.5 Hz, 14.6 Hz, 1H), 2.37 (s, 3H).

¹³C NMR (CDCl₃, 100 MHz) δ_{c} 195.3 (1C), 167.8 (1C), 160.4 (1C), 139.5 (1CH), 130.8 (1CH), 127.1 (1C), 124.5 (1CH), 119.6 (1CH₂), 119.1 (1C), 108.2 (1CH), 77.1 (1C), 53.0 (1CH₃), 36.4 (1CH₂), 29.2 (1CH₃), 20.3 (1CH₃).

IR υₘₐₓ (thin film, cm⁻¹): 2952, 2920, 1737, 1686, 1620, 1575, 1504.

HRMS m/z (ESI+) calculated for C₁₅H₁₇NO₃[M+H]⁺; 260.1279, observed 260.1281.

### Methyl 1-methyl-3-oxo-2-(propa-1,2-dien-1-yl)indoline-2-carboxylate 3ab

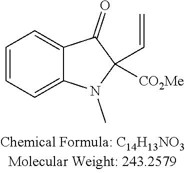 [SMILES: C=CC1(C(=O)OC)C(=O)c2ccccc2N1C]

According to general procedure 2, dimethyl 2-(methyl(prop-2-yn-1-yl)amino)malonate (66 mg, 0.33 mmol, 1 eq) was reacted with 2-(trimethylsilyl)phenyl trifluoromethanesulfonate (149 mg, 0.50 mmol, 1.5 eq) to yield compound 3ab (56 mg, 70%) as a bright yellow solid after silica gel column chromatography (Hexane: tOAc, 7:1).

mp 87-89° C.

¹H NMR (CDCl₃, 400 MHz) δ_{H} 7.57 (d, J 7.7 Hz, 1H), 7.48-7.52 (m, 1H), 6.79 (d, J 8.3 Hz, 1H), 6.76 (app t, J 7.5 Hz, 1H), 5.78 (t, J 6.7 Hz, 1H), 4.98 (dd, J 6.7 Hz, 11.5 Hz, 1H), 4.14 (dd, J 6.7 Hz, 11.5 Hz, 1H), 3.77 (s, 3H), 3.01 (s, 3H).

¹³C NMR (CDCl₃, 100 MHz) δ_{c} 207.4 (1C), 193.5 (1C), 166.7 (1C), 161.4 (1C), 138.1 (1CH), 125.8 (1CH), 117.9 (1CH), 117.8 (1C), 108.4 (1CH), 87.8 (1CH), 79.3 (1CH₂), 76.2 (1CH), 53.3 (1CH₃), 30.0 (1CH₃).

IR υₘₐₓ(thin film, cm⁻¹): 3060, 3024, 2953, 2924, 2888, 2836, 1737, 1698, 1611, 1486.

HRMS m/z (ESI+) calculated for C₁₄H₁₃NO₃[M+H]⁺, 244.0968, observed 244.0975.

### Methyl 4-methoxy-1-methyl-3-oxo-2-(propa-1,2-dien-1-yl)indoline-2-carboxylate 3bb

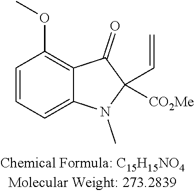 [SMILES: C=CC1(C(=O)OC)C(=O)c2c(OC)cccc2N1C]

According to general procedure 2, dimethyl 2-(methyl(prop-2-yn-1-yl)amino)malonate (132 mg, 0.67 mmol, 1 eq) was reacted with 3-methoxy-2-(trimethylsilyl)phenyl trifluoromethanesulfonate (327 mg, 1.0 mmol, 1.5 eq) to yield compound 3bb (147 mg, 81%) as a bright yellow solid after silica gel column chromatography (Toluene : EtOAc, 10:1).

mp: 135-137° C.

¹H NMR (CDCl₃, 400 MHz) δH 7.40 (app. t, J 8.2 Hz, 1H), 6.34 (d, J 8.2 Hz, 1H), 6.16 (d, J 8.1 Hz, 1H), 5.79 (t, J 6.7 Hz, 1H), 4.96 (dd, J 6.7 Hz, 11.5 Hz, 1H), 4.86 (dd, J 6.7 Hz, 11.5 Hz, 1H), 3.89 (s, 3H), 3.75 (s, 3H), 2.97 (s, 3H).

¹³C NMR (CDCl₃, 100 MHz) δC 207.2 (1C), 190.7 (1C), 166.7 (1C), 162.7 (1C), 159.8 (1C), 139.7 (1CH), 106.7 (1CH), 100.6 (1C), 99.2 (1CH), 88.2 (1CH), 79.2 (1CH₂), 76.3 (1C), 55.8 (1CH₃), 53.2 (1CH₃), 30.2 (1CH₃).

IR υₘₐₓ(thin film, cm⁻¹): 2952, 1732, 1599, 1579, 1496.

HRMS m/z (ESI+) calculated for C₁₅H₁₅NO₄[M+H]⁺; 274.1074, observed 274.1082.

### Methyl 1,4-dimethyl-3-oxo-2-(propa-1,2-dien-1-yl)indoline-2-carboxylate 3cb

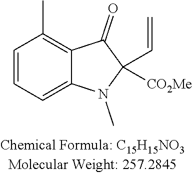 [SMILES: C=CC1(C(=O)OC)C(=O)c2c(C)cccc2N1C]

According to general procedure 2, dimethyl 2-(methyl(prop-2-yn-1-yl)amino)malonate (106 mg, 0.53 mmol, 1 eq) was reacted with 3-methyl-2-(trimethylsilyl)phenyl trifluoromethanesulfonate (250 mg, 0.8 mmol, 1.5 eq) to yield compound 3cb (87 mg, 64%) as a bright yellow solid after silica gel column chromatography (Petrol : Acetone 12:1).

mp: 115-117° C.

¹H NMR (CDCl₃, 400 MHz) δH 7.33 (dd, J 7.6 Hz, 8.0 Hz, 1H), 6.59 (d, J 8.3 Hz, 1H), 6.50 (d, J 7.3 Hz, 1H), 5.79 (t, J 6.7 Hz, 1H), 4.97 (dd, J 6.7 Hz, 11.4 Hz, 1H), 4.86 (dd, J 6.7 Hz, 11.4 Hz, 1H), 3.76 (s, 3H), 2.98 (s, 3H), 2.52 (s, 3H).

¹³C NMR (CDCl₃, 100 MHz) δC 207.2 (1C), 193.8 (1C), 166.9 (1C), 161.9 (1C), 141.3 (1CH), 137.4 (1CH), 119.5 (1CH), 116.0 (1C), 105.5 (1C), 88.3 (1CH), 79.2 (1CH₂), 76.2 (1C), 53.3 (1CH₃), 30.1 (1CH₃), 18.3 (1CH₃).

IR υₘₐₓ (thin film, cm⁻¹): 2948, 1732, 1687, 1601, 1494.

HRMS m/z (ESI+) calculated for C₁₅H₁₅NO₃[M+H]⁺; 258.1125, observed 258.1122.

### Methyl 3-methyl-1-oxo-2-(propa-1,2-dien-1-yl)-2,3-dihydro-1H-benzo[e]indole-2-carboxylate 3db

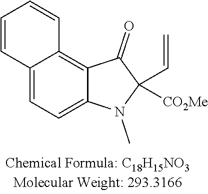 [SMILES: C=CC1(C(=O)OC)C(=O)c2c(ccc3ccccc23)N1C]

According to general procedure 2, dimethyl 2-(methyl(prop-2-yn-1-yl)amino)malonate (132 mg, 0.67 mmol, 1 eq) was reacted with 1-(trimethylsilyl)naphthalen-2-yl trifluoromethanesulfonate (347 mg, 1.0 mmol, 1.5 eq) to yield compound 3db (150 mg, 77%) as a bright yellow solid after silica gel column chromatography (Hexane:EtOAc 9:1).

mp: 145-147° C.

¹H NMR (CDCl₃, 400 MHz) δ_{H} 8.65 (d, J 8.3 Hz, 1H), 7.95 (d, J 9.0 Hz, 1H), 7.71 (d, J 8.1 Hz, 1H) 7.57 (ddd, J 1.2 Hz, 7.1 Hz, 8.3 Hz, 1H), 7.31 (ddd, J 1.1 Hz, 6.9 Hz, 8.1 Hz, 1H), 7.07 (d, J 9.1 Hz, 1H), 5.89 (t, J 6.7 Hz, 1H), 4.99 (dd, J 6.7 Hz, 11.4 Hz, 1H), 4.87 (dd,J 6.7 Hz, 11.4 Hz, 1H), 3.79 (s, 3H), 3.14 (s, 3H).

¹³C NMR (CDCl₃, 100 MHz) δ_{C} 207.1 (1C), 191.8 (1C), 166.5 (1C), 163.9 (1C), 140.2 (1CH), 130.9 (1CH), 130.1 (1CH), 128.6 (1CH), 127.2 (1CH), 123.7 (1CH), 122.6 (1C), 110.1 (1C), 107.9 (1C), 88.0 (1CH), 79.3 (1C), 76.8 (1CH₂), 53.4 (1CH₃), 30.3 (1CH₃).

HRMS m/z (ESI+) calculated for C₁₈H₁₅NO₃[M+H]⁺; 294.1125, observed 294.1133.

### Methyl 5-methoxy-1-methyl-3-oxo-2-(propa-1,2-dien-1-yl)indoline-2-carboxylate 3eb

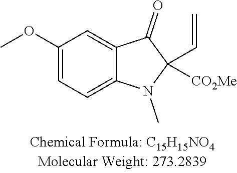 [SMILES: C=CC1(C(=O)OC)C(=O)c2cc(OC)ccc2N1C]

According to general procedure 2, dimethyl 2-(methyl(prop-2-yn-1-yl)amino)malonate (132 mg, 0.67 mmol, 1 eq) was reacted with 4-methoxy-2-(trimethylsilyl)phenyl trifluoromethanesulfonate (327 mg, 1.0 mmol, 1.5 eq) to yield compounds 3eb (81 mg, 45%) as a bright yellow solid and 3eb′ (45 mg, 25%) as a bright yellow solid after silica gel column chromatography (Petrol:EtOAc 5:1).

mp98-100° C.

¹H NMR (CDCl₃, 400 MHz) δH 7.19, (dd, J 2.7 Hz, 8.9 Hz, 1H), 7.02 (d, J 2.7 Hz, 1H), 6.77 (d, J 8.9 Hz, 1H), 5.78 (t, J 6.7 Hz, 1H), 4.97 (dd, J 6.7 Hz, 11.5 Hz, 1H), 4.88 (dd, J 6.7 Hz, 11.5 Hz, 1H), 3.77 (s, 3H), 3.76 (s, 3H), 2.98 (s, 3H).

¹³C NMR (CDCl₃, 100 MHz) δC 207.4 (1C), 193.4 (1C), 166.7 (1C), 157.7 (1C), 152.5 (1C), 128.7 (1CH), 117.6 (1C), 109.8 (1CH), 105.6 (1CH), 87.8 (1CH), 79.1 (1CH₂), 76.8 (1C), 55.8 (1CH₃), 53.2 (1CH₃), 30.2 (1CH₃).

IR υₘₐₓ (thin film, cm⁻¹): 2927, 1743, 1674, 1609, 1574, 1488.

HRMS m/z (ESI+) calculated for C₁₅H₁₅NO₄[M+H]⁺; 274.1074, observed 274.1069.

### Methyl 6-methoxy-1-methyl-3-oxo-2-(propa-1,2-dien-1-yl)indoline-2-carboxylate 3eb′

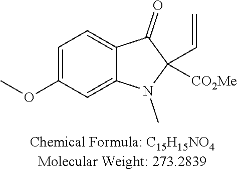 [SMILES: C=CC1(C(=O)OC)C(=O)c2ccc(OC)cc2N1C]

According to general procedure 2, dimethyl 2-(methyl(prop-2-yn-1-yl)amino)malonate (132 mg, 0.67 mmol, 1 eq) was reacted with 4-methoxy-2-(trimethylsilyl)phenyl trifluoromethanesulfonate (327 mg, 1.0 mmol, 1.5 eq) to yield compounds 3eb (81 mg, 45%) as a bright yellow solid and 3eb′ (45 mg, 25%) after silica gel column chromatography (Petrol:EtOAc 5:1).

mp: 105-107° C.

¹H NMR (CDCl₃, 400 MHz) δ_{H} 7.48 (d, J 8.6 Hz, 1H), 6.33 (dd, J 2.0 Hz, 8.6 Hz, 1H), 6.16 (d, J 2.0 Hz, 1H), 5.80 (t, J 6.7 Hz, 1H), 4.97 (dd, J 6.7 Hz, 11.5 Hz, 1H), 4.87 (dd, J 6.7 Hz, 11.5 Hz, 1H), 3.88 (s, 3H), 3.76 (s, 3H), 2.99 (s, 3H).

¹³C NMR (CDCl₃, 100 MHz) δ_{C} 207.2 (1C), 190.9 (1C), 168.6 (1C), 166.8 (1C), 163.5 (1C), 127.3 (1CH), 111.2 (1C), 107.4 (1CH), 91.2 (1CH), 88.2 (1CH), 79.2 (1CH₂), 76.8 (1C), 55.6 (1CH₃), 53.3 (1CH₃), 30.1 (1CH₃).

IR υₘₐₓ (thin film, cm⁻¹): 2950, 2835, 1734, 1677, 1602, 1580, 1500.

HRMS m/z (ESI+) calculated for C₁₅H₁₅NO₄[M+H]⁺; 274.1074, observed 274.1070.

### Methyl 1-methyl-3-oxo-2-(propa-1,2-dien-1-yl)-2,3-dihydro-1H-benzo[f]indole-2-carboxylate 3gb

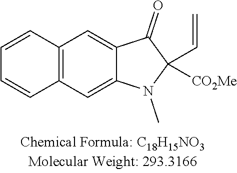 [SMILES: C=CC1(C(=O)OC)C(=O)c2cc3ccccc3cc2N1C]

According to general procedure 2, dimethyl 2-(methyl(prop-2-yn-1-yl)amino)malonate (132 mg, 0.67 mmol, 1 eq) was reacted with 3-(trimethylsilyl)naphthalen-2-yl trifluoromethanesulfonate (347 mg, 1.0 mmol, 1.5 eq) to yield compound 3gb (70 mg, 36%) as an orange solid after silica gel column chromatography (toluene, 100%).

mp 145-147° C.

¹H NMR (CDCl₃, 400 MHz) δ_{H} 8.16 (s, 1H), 7.76 (d, J 8.3 Hz, 1H), 7.64 (d, J 8.3 Hz, 1H), 7.45 (ddd, J 1.0 Hz, 6.9 Hz, 8.1 Hz, 1H), 7.21 (app. t, J 7.5 Hz, 1H), 6.90 (s, 1H), 5.83 (t, J 6.7 Hz, 1H), 4.99 (dd, J 6.7 Hz, 11.5 Hz, 1H), 4.87 (dd, J 6.7 Hz, 11.5 Hz, 1H), 3.77 (s, 3H), 3.07 (s, 3H).

¹³C NMR (CDCl₃, 100 MHz) δ_{C} 207.6 (1C), 194.3 (1C), 167.2 (1C), 154.7 (1C), 140.2 (1C), 130.8 (1CH), 129.8 (1CH), 127.5 (1CH), 127.2 (1C), 126.4 (1CH), 122.9 (1CH), 120.7 (1C), 101.2 (1CH), 88.1 (1CH), 79.5 (1CH₂), 76.3 (1C), 53.3 (1CH₃), 30.3 (1CH₃).

IR 80 ₘₐₓ (thin film, cm⁻¹): 2918, 1744, 1708, 1625, 1504.

HRMS m/z (ESI+) calculated for C₁₈H₁₅NO₃NO₃[M+H]⁺; 294.1125, observed 294.1122.

Methyl 5,6-dimethoxy-1-methyl-3-oxo-2-(propa-1,2-dien-1-yl)indoline-2-carboxylate 3hb

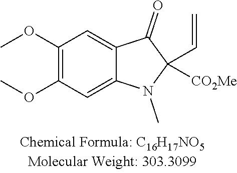 [SMILES: C=CC1(C(=O)OC)C(=O)c2cc(OC)c(OC)cc2N1C]

According to general procedure 2, dimethyl 2-(methyl(prop-2-yn-1-yl)amino)malonate (132 mg, 0.67 mmol, 1 eq) was reacted with 4,5-dimethoxy-2-(trimethylsilyl)phenyl trifluoromethanesulfonate (357 mg, 1.0 mmol, 1.5 eq) to yield compound 3hb (148 mg, 74%) as a bright yellow oil after silica gel column chromatography (Petrol:Acetone 3:1).

¹H NMR (CDCl₃, 400 MHz) δH 6.97 (s, 1H), 6.24 (s, 1H), 5.79 (t, J 6.7 Hz, 1H), 4.96 (dd, J 6.7 Hz, 11.4 Hz, 1H), 4.87 (dd,J 12 Hz, 7 Hz, 1H), 3.98 (s, 3H), 3.81 (s, 3H), 3.77 (s, 3H), 3.00 (s, 3H).

¹³C NMR (CDCl₃, 100 MHz) δC 207.2 (1C), 191.1 (1C), 166.9 (1C), 159.6 (1C), 159.3 (1C), 143.3 (1C), 108.7 (1C), 105.3 (1CH), 90.7 (1CH), 88.1 (1CH), 79.0 (1CH₂), 76.6 (1C), 56.24 (1CH₃), 56.20 (1CH₃), 53.3 (1CH₃), 30.2 (1CH₃).

IR υₘₐₓ (thin film, cm⁻¹): 2952, 1737, 1674, 1620, 1578, 1497.

HRMS m/z (ESI+) calculated for C₁₆H₁₇NO₅ [M+H]⁺; 304.1179, observed 304.1173.

### Methyl 4-methoxy-1-methyl-2-(2-methylbut-3-en-2-yl)-3-oxoindoline-2-carboxylate 3bc

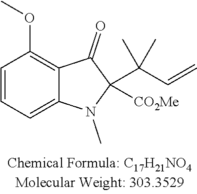 [SMILES: C=CC(C)(C)C1(C(=O)OC)C(=O)c2c(OC)cccc2N1C]

According to general procedure 2, dimethyl 2-(methyl(3-methylbut-2-en-1-yl)amino)malonate (0.541 mmol, 1 eq, 162 mg) was reacted with 3-methoxy-2-(trimethylsilyl)phenyl trifluoromethanesulfonate (267 mg, 0.81 mmol, 1.5 eq) to yield compounds 3bc (108 mg, 66%) as a bright yellow solid and 3bc′ (8 mg, 6%) as a bright yellow oil, after silica gel column chromatography (Toluene:EtOAc 10:1).

mp: 86-88° C.

¹H NMR (CDCl₃, 400 MHz) δH 7.39 (app. t, J 8.2 Hz, 1H), 6.35 (d, J 8.2 Hz, 1H), 6.30 (dd, J 10.8 Hz, 17.5 Hz, 1H), 6.19 (d, J 9 Hz, 1H), 5.08 (dd, J 1.2 Hz, 17.3 Hz, 1H), 5.04 (dd, J 1.2 Hz, 10.7 Hz, 1H), 3.91 (s, 3H), 3.65 (s, 3H), 2.98 (s, 3H), 1.40 (s, 3H), 1.18 (s, 3H).

¹³C NMR (CDCl₃, 100 MHz) δC 193.8 (1C), 167.2 (1C), 163.8 (1C), 159.0 (1C), 143.8 (1CH), 138.9 (1CH), 113.4 (1CH₂), 109.5 (1C), 101.4 (1CH), 99.6 (1CH), 80.5 (1C), 55.8 (1CH₃), 52.2 (1CH₃), 44.2 (1C), 34.0 (1CH₃), 22.6 (1CH₃), 21.5 (1CH₃).

HRMS m/z (ESI+) calculated for C₁₂H₂₁NO₄ [M+H]⁺; 304.1543, observed 304.1549.

### Methyl 4-methoxy-1-methyl-2-(3-methylbut-2-en-1-yl)-3-oxoindoline-2-carboxylate 3bc′

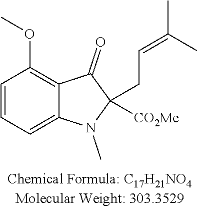 [SMILES: COC(=O)C1(CC=C(C)C)C(=O)c2c(OC)cccc2N1C]

According to general procedure 2, dimethyl 2-(methyl(3-methylbut-2-en-1-yl)amino)malonate (0.541 mmol, 1 eq, 162 mg) was reacted with 3-methoxy-2-(trimethylsilyl)phenyl trifluoromethanesulfonate (267 mg, 0.81 mmol, 1.5 eq) to yield compounds 3bc (108 mg, 66%) as a bright yellow solid and 3bc′ (8 mg, 6%) as a bright yellow oil, after silica gel column chromatography (Toluene:EtOAc 10:1).

¹H NMR (CDCl₃, 400 MHz) δH 7.40 (app. t, J 8.2 Hz, 1H), 6.34 (d, J 8.3 Hz, 1H), 6.15 (d, J, 8.1 Hz, 1H), 4.72-4.76 (m, 1H), 3.90 (s, 3H), 3.70 (s, 3H), 3.01 (dd, J 7.2 Hz, 15.5 Hz, 1H), 2.94 (s, 3H), 2.80 (dd, J 7.0 Hz, 15.5 Hz, 1H), 1.64 (s, 3H), 1.56 (s, 3H).

¹³C NMR (CDCl₃, 100 MHz) 6C 192.8 (1C), 168.1 (1C), 163.1 (1C), 159.2 (1C), 139.4 (1CH), 135.8 (1CH), 116.0 (1CH), 108.2 (1CH), 100.5 (1CH), 98.8 (1CH), 77.2 (1C), 55.80 (1CH₃), 52.94 (1CH₃), 30.9 (1CH₂), 29.4 (1CH₃), 25.8 (1CH₃), 18.2 (1CH₃).

IR υₘₐₓ(thin film, cm⁻¹): 2963, 1741, 1698, 1609, 1498.

HRMS m/z (ESI+) calculated for C₁₂H₂₁NO₄[M+H]⁺; 304.1543, observed 304.1549.

### Methyl 2-(but-3-en-2-yl)-4-methoxy-1-methyl-3-oxoindoline-2-carboxylate 3bd

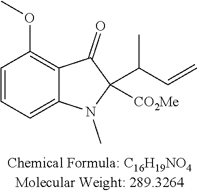 [SMILES: C=CC(C)C1(C(=O)OC)C(=O)c2c(OC)cccc2N1C]

According to general procedure 2, (E)-2-(but-2-en-1-yl(methyl)amino)malonate (143 mg, 0.67 mmol, 1 eq) was reacted with 3-methoxy-2-(trimethylsilyl)phenyl trifluoromethanesulfonate (327 mg, 1.0 mmol, 1.5 eq) to yield compound 3bd (123 mg, 64%) as a bright yellow solid after silica gel column chromatography (Toluene:EtOAc 8:1). mp: 89-91° C.

¹H NMR (CDCl₃, 300 MHz) δH 7.40 (app. t, J 8.2 Hz, 1H), 6.35 (d, J 8.2 Hz, 1H), 6.15 (d, J 8.1 Hz, 1H), 5.94 (ddd, J 6.5 Hz, 10.5 Hz, 17.2 Hz, 1H), 5.22 (app. dm, J 17.2 Hz, 1H), 5.14 (app. dm, J 10.5 Hz, 1H), 3.91 (s, 3H), 3.73 (s, 3H), 3.41-3.50 (m, 1H), 3.03 (s, 3H), 0.94 (d, J 6.8 Hz, 3H).

¹³C NMR (CDCl₃, 100 MHz) δC 191.6 (1C), 167.2 (1C), 163.4 (1C), 159.2 (1C), 139.5 (1CH), 138.6 (1CH), 117.0 (1CH₂), 108.0 (1C), 100.7 (1CH), 98.9 (1CH), 80.9 (1C), 55.8 (1CH₃), 52.8 (1CH₃), 42.2 (1CH), 31.9 (1CH₃), 12.3 (1CH₃).

IR υₘₐₓ (thin film, cm⁻¹): 2976, 2953, 1735, 1688, 1601, 1586, 1498.

HRMS m/z (ESI+) calculated for C₁₆H₁₉NO₄[M+H]⁺; 290.1387, observed 290.1385.

### Methyl 1-methyl-2-(2-methylenecyclohexyl)-3-oxoindoline-2-carboxylate 3ae

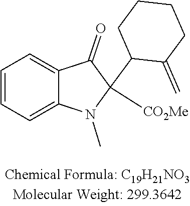 [SMILES: C=C1CCCCC1C1(C(=O)OC)C(=O)c2ccccc2N1C]

According to general procedure 2, 2-((cyclohex-1-en-1-ylmethyl)(methyl)amino)malonate (169 mg, 0.67 mmol, 1 eq) was reacted with 2-(trimethylsilyl)phenyl trifluoromethanesulfonate (297 mg, 1.0 mmol, 1.5 eq) to yield compound 3ae (105 mg, 48%) as a bright yellow oil after silica gel column chromatography (Petrol:EtOAc, 10:1).

¹H NMR (CDCl₃, 400 MHz) δH 7.56 (app. dm, J 7.8 Hz, 1H), 7.48 (ddd, J 1.3 Hz, 7.1 Hz, 8.4 Hz, 1H), 6.81 (d, J 8.4, 1H), 6.72 (app. t, J 7.4 Hz, 1H), 4.79 (s, 1H), 4.51 (s, 1H), 3.70 (s, 3H), 3.31-3.35 (m, 1H), 3.19 (s, 3H), 2.29-2.34 (m, 1H), 2.13 (td, J 4.6 Hz, 12.5 Hz, 1H), 1.68-1.78 (m, 2H), 1.29-1.45 (m, 4H).

¹³C NMR (CDCl₃, 100 MHz) δ_{C} 195.3 (1C), 167.9 (1C), 161.8 (1C), 148.1 (1C), 138.0 (1CH), 125.0 (1CH), 118.5 (1C), 117.4 (1CH), 108.4 (1CH), 107.4 (1CH₂), 79.3 (1C), 53.0 (1CH₃), 48.7 (1CH), 37.8 (1CH₂), 30.9 (1CH₃), 28.0 (2CH₂), 25.7 (1CH₂).

IR υₘₐₓ (thin film, cm⁻¹): 1733, 1705.

HRMS m/z (ESI+) calculated for C₁₈H₂₁NO₃[M+H]⁺; 300.1594, observed 300.1614.

### Methyl 3-oxo-1-(prop-2-yn-1-yl)-2-(propa-1,2-dien-1-yl)indoline-2-carboxylate 3af

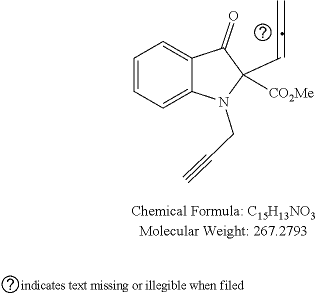 [SMILES: C#CCN1c2ccccc2C(=O)C1(C=C)C(=O)OC]

According to general procedure 2, dimethyl 2-(di(prop-2-yn-1-yl)amino)malonate (583 mg, 2.61 mmol, 1 eq) was reacted with 2-(trimethylsilyl)phenyl trifluoromethanesulfonate (1.23 g, 4.11 mmol, 1.5 eq) to yield compound 3af (118 mg, 17%) as a bright yellow oil after silica gel column chromatography (pentane:acetone, 9:1).

¹H NMR (CDCl₃, 400 MHz) δH 7.62 (d, J 7.7 Hz, 1H), 7.55 (ddd, J 1.3 Hz, 7.3 Hz, 8.4 Hz, 1H), 7.02 (d, J 8.3 Hz, 1H), 6.86 (app. t, J 7.4 Hz, 1H), 5.76 (t J 6.7 Hz, 1H), 5.02 (dd, J 6.7 Hz, 11.7 Hz, 1H), 4.94 (dd, J 6.7 Hz, 11.7 Hz, 1H), 4.22 (dd, J 2.5 Hz, 18.2 Hz, 1H), 4.16 (dd, J 2.5 Hz, 18.2 Hz, 1H), 3.76 (s, 3H), 2.28 (t, J 2.5 Hz, 1H).

¹³C NMR (CDCl₃, 100 MHz) δ_{C} 207.6 (1C), 193.1 (1C), 166.9 (1C), 159.5 (1C), 137.9 (1CH), 125.7 (1CH), 119.0 (1CH), 118.7 (1C), 109.6 (1CH), 87.8 (1CH), 79.9 (1CH₂), 78.1 (1C), 75.8 (1C), 72.5 (1CH), 53.2 (1CH₃), 33.9 (1CH₂).

HRMS m/z (ESI+) calculated for C₁₆H₁₃NO₃[M+H]⁺; 268.0974, observed 268.0972.

### Methyl 2-allyl-3-oxo-1-(prop-2-yn-1-yl)indoline-2-carboxylate 3ag

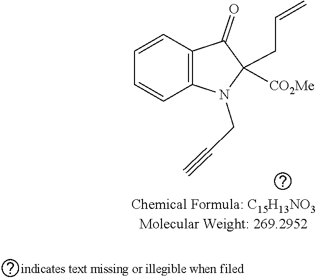 [SMILES: C#CCN1c2ccccc2C(=O)C1(CC=C)C(=O)OC]

According to general procedure 2, dimethyl 2-(allyl(prop-2-yn-1-yl)amino)malonate (216 mg, 0.96 mmol, 1 eq) was reacted with 2-(trimethylsilyl)phenyl trifluoromethanesulfonate (430 mg, 1.44 mmol, 1.5 eq) to yield compound 3ag (59 mg, 23%) as a bright yellow oil after silica gel column chromatography (pentane:acetone, 20:1 to 10:1).

¹H NMR (CDCl₃, 400 MHz) δ_{H} 7.60 (d, J 7.7 Hz, 1H), 7.52-7.56 (m, 1H), 6.99 (d, J 8.3 Hz, 1H), 6.84 (app t, J 7.4 Hz, 1H), 5.49-5.59 (m, 1H), 5.20 (dd, J 1.3 Hz, 17.0 Hz, 1H), 5.01 (d, J 9.9 Hz, 1H), 4.21 (dd, J 2.5 Hz, 18.4 Hz, 1H), 4.14 (dd, J 2.5 Hz, 18.4 Hz, 1H), 3.71 (s, 3H), 2.95-3.05 (m, 2H), 2.29 (t, J 2.4 Hz, 1H).

¹³C NMR (CDCl₃, 100 MHz) δ_{C} 194.8 (1C), 167.7 (1C), 159.9 (1C), 137.9 (1CH), 130.4 (1CH), 125.2 (1CH), 120.3 (1CH₂), 119.7 (1C), 118.8 (1CH), 109.3 (1CH), 78.0 (1C), 76.7 (1C), 72.6 (1CH), 53.1 (1CH₃), 37.0 (1CH₂), 33.4 (1CH₂).

IR υₘₐₓ (thin film, cm⁻¹): 3263, 3058, 2950, 2918, 2850, 1740, 1687,1609, 1484.

HRMS m/z (ESI⁺) calculated for C₁₆H₁₅NO₃(M+H)⁺ expected 270.1125, found 270.1124.

Methyl 2-allyl-1-benzyl-4-methoxy-3-oxoindoline-2-carboxylate 3bh

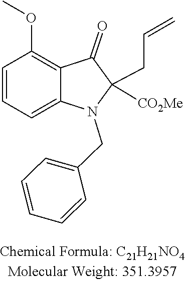 [SMILES: C=CCC1(C(=O)OC)C(=O)c2c(OC)cccc2N1Cc1ccccc1]

According to general procedure 2, dimethyl 2-(allyl(benzyl)amino)malonate (184 mg, 0.67 mmol, 1 eq) was reacted with 3-methoxy-2-(trimethylsilyl)phenyl trifluoromethanesulfonate (327 mg, 1.0 mmol, 1.5 eq) to yield compound 3bh (17 mg, 16%) as a bright yellow oil after silica gel column chromatography (Petrol:EtOAc, 10:1).

¹H NMR (CDCl₃, 400 MHz) δH 7.24-7.35 (m, 6H), 6.24 (d, J 8.2 Hz, 1H), 6.21 (d, J 8.1 Hz, 1H), 5.44-5.52 (m, 1H), 5.11 (ddd, J 1.5 Hz, 3.0 Hz, 17.0 Hz, 1H), 4.98 (app. dm, J 10.1 Hz, 1H), 4.58 (d, J 16.7 Hz, 1H), 4.53 (d, J 16.7 Hz, 1H), 3.92 (s, 3H), 3.53 (s, 3H), 3.02 (app. dd, J 6.7 Hz, 14.8 Hz, 1H), 2.95 (app. dd, J 7.5 Hz, 14.8 Hz, 1H).

¹³C NMR (CDCl₃, 100 MHz) δC 192.9 (1C), 168.1 (1C), 163.1 (1C), 159.2 (1C), 139.4 (1CH), 136.9 (1C), 130.6 (1CH), 128.6 (2CH), 127.4 (1CH), 127.1 (2CH), 120.1 (1CH₂), 108.8 (1C), 101.9 (1CH), 99.7 (1CH), 77.1 (1C), 55.8 (1CH₃), 52.7 (1CH₃), 48.5 (1CH₂), 36.8 (1CH₂).

IR υₘₐₓ (thin film, cm⁻¹): 2926, 1740, 1690, 1599, 1493.

HRMS m/z (ESI+) calculated for C₂₁H₂₁NO₄[M+H]⁺; 352.1549, observed 352.1531.

Methyl 2-allyl-5,6-dimethoxy-1-methyl-3-oxoindoline-2-carboxylate 90f

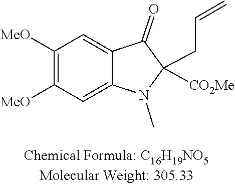 [SMILES: C=CCC1(C(=O)OC)C(=O)c2cc(OC)c(OC)cc2N1C]

According to general procedure 3, dimethyl 2-(allyl(methyl)amino)malonate was reacted with 4,5-dimethoxy-2-(trimethylsilyl)phenyl trifluoromethanesulfonate to yield the title compound after column chromatography (6:4 v/v petrol:EtOAc) as a fluorescent yellow solid

Rf=0.17 (6:4 v/v petrol:EtOAc); mp: 100-101° C.;

¹H NMR (CDCl₃, 400 MHz) δH 2.86 (dd, J 7.5 Hz 14.5 Hz, 1H), 3.00 (s, 3H), 3.07 (dd, J 6.6 Hz 14.5 Hz, 1H), 3.73 (s, 3H), 3.82 (s, 3H), 3.99 (s, 3H), 4.99 (dd, J 1.5 Hz 10.0 Hz, 1H), 5.15 (dd, J 1.5 Hz 17.1 Hz, 1H), 5.42 (ddt, J 7.1 Hz 10.0 Hz 17.1 Hz, 1H), 6.23 (s, 1H), 6.99 (s, 1H).

¹³C NMR (CDCl₃, 100 MHz) δC 29.5 (CH₃), 36.5 (CH₂), 53.1 (CH₃), 56.2 (CH₃), 56.3 (CH₃), 77.2 (C), 90.6 (CH), 104.8 (CH), 110.2 (C), 119.4 (CH₂), 131.0 (CH), 143.2 (C), 159.2 (C), 159.8 (C), 168.0 (C), 192.5 (C).

IR υₘₐₓ (thin film, cm⁻¹): 1732 (C═O ester), 1661 (C═O aryl), 1347 (C—N aryl), 1218 (C—O aryl);

UV-Vis (EtOH) λmax (nm), Σ(M⁻¹cm⁻¹) 224 (13364), 251 (15354), 283 (11763), 418 (6938);

HRMS m/z (ESI+) calculated for C₁₆H₁₉NO₅[M+H]⁺306.1336, found 306.1335.

### Methyl 2-allyl-1-methyl-3-oxo-2,3-dihydro-1H-benzo[f]indole-2-carboxylate

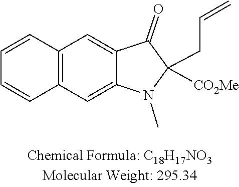 [SMILES: C=CCC1(C(=O)OC)C(=O)c2cc3ccccc3cc2N1C]

According to general procedure 3, dimethyl 2-(allyl(methyl)amino)malonate was reacted with 3-(trimethylsilyl)-2-naphthyl trifluoromethanesulfonate to yield the title compound after column chromatography (9.5:0.5 v/v petrol:EtOAc) as a red solid.

Rf=0.24 (9.5:0.5 v/v petrol:EtOAc)

mp: 94-96° C.

¹H NMR (CDCl₃, 400 MHz) δH 2.98 (dd, J 7.3 Hz 14.7 Hz, 1H), 3.07 (s, 3H), 3.12 (dd, J 7.0 Hz 14.7 Hz, 1H), 3.73 (s, 3H), 4.98 (dd, J 1.1 Hz, 10.1 Hz, 1H), 5.18 (dd, J 1.1 Hz 17.0 Hz, 1H), 5.44 (ddt, J 7.1 Hz 10.1 Hz 17.0 Hz, 1H), 6.92 (s, 1H), 7.23 (t, J 7.5 Hz, 1H), 7.47 (t, J 7.6 Hz, 1H), 7.66 (d, J 8.4 Hz, 1H), 7.79 (d, J 8.2 Hz, 1H), 8.16 (s, 1H).

¹³C NMR (CDCl₃, 100 MHz) δC 29.3 (CH₃), 36.3 (CH₂), 53.0 (CH₃), 76.8 (C), 101.2 (CH), 119.9 (CH₂), 121.9 (C), 122.9 (CH), 126.4 (CH), 126.7 (CH), 127.1 (C), 129.7 (CH), 130.6 (CH), 130.9 (CH), 140.2 (C), 155.1 (C), 168.2 (C), 196.6 (C).

IR υₘₐₓ (thin film, cm⁻¹): 1744 (C═O ester), 1714 (C═O aryl), 1327 (C-Naryl)

UV-Vis (EtOH) λmax (nm), Σ(M⁻¹cm⁻¹) 251 (58250), 258 (58250), 325 (5016), 484 (1298); m/z

HRMS m/z (ESI+) calculated for C₁₈H₁₇NO₃[M+Na]+; 318.1101, found 318.1100.

### N-(4-Trifluoromethoxy)phenyl)ethane-1,2-diamine

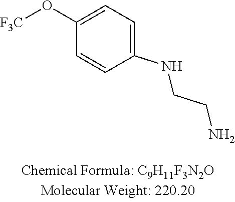 [SMILES: NCCNc1ccc(OC(F)(F)F)cc1]

A solution of 4-trifluoromethoxyaniline (2.7 mL, 20.00 mmol, 2.0 equiv.) and 2-bromoethylamine hydrobromide (2.05 g, 10.00 mmol, 1.0 equiv.) in 10.0 mL toluene was stirred at reflux overnight. Once cooled the reaction mixture was diluted with 13.0 mL 30% NaOH solution and washed twice with toluene. The organic layers were combined, dried over Na2SO4, filtered and concentrated under reduced pressure to yield a crude deep orange oil. The crude oil was purified via column chromatography using 9:1 DCM:MeOH with 1.25% NH4OH to yield N-(4-trifluoromethoxy)phenyl)ethane-1,2-diamine (203, 1.43 g, 6.50 mmol, 65%) as a pale orange oil; Rf 0.42 (9:1 DCM:MeOH with 1.25% NH4OH).

IR vmax/cm-1 3317, 3040, 2938, 1614, 1514, 1252; 1H NMR (400 MHz, CDCl₃) δ; 7.03 (2H, d, J=9.0 Hz, H-3′ and H-5′), 6.58 (2H, d, J=9.0 Hz, H-2′ and H-6′), 4.23 (1H, bs, NH), 3.16 (2H, t, J=6.0 Hz, H-1), 2.97 (2H, t, J=6.0 Hz, H-2), 1.70 (2H, bs, NH2); 13C NMR (100MHz, CDCl₃) δ; 40.8 (C-2), 46.3 (C-1), 113.0 (C-2′ and C-6′), 122.4 (C-3′ and C-5′), 140.4 (Ar(OCF3)), 147.2 (ArC), 147.5 (ArC); MS m/z [M+H]+C9H12F3N20 requires 221.08, found 221.10.

### 3-(2-azidoethyl)-6-(trifluoromethoxy)benzo[d]thiazol-2(3H)-imine 15

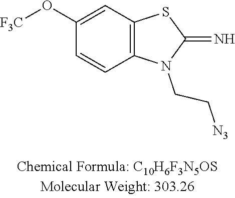 [SMILES: [N-]=[N+]=NCCn1c(=N)sc2cc(OC(F)(F)F)ccc21]

In a dry 50-mL RBF, to a solution of N¹-(4-(trifluoromethoxy)phenyl)ethane-1,2-diamine (0.83 g, 3.75 mmol, 1 eq), K₂CO₃ (1.21 g, 8.75 mmol, 2.3 eq) and CuSO₄.5H₂O (9 mg, 0.04 mmol, 0.01 eq) in MeOH (20 mL) stirred under N₂, was added imidazole-1-sulfonyl azide hydrochloride (0.94 g, 4.50 mmol, 1.2 eq) in 3 portions. The reaction mixture was stirred at r.t. for 2 h and then diluted with H₂O (60 mL), acidified with conc. HCl and extracted with EtOAc (2×80 mL). The combined organic layers were dried over MgSO₄, filtered and concentrated under reduced pressure to yield 14 as a crude orange oil (0.95 g, 100% pure by ¹H NMR). In a dry 50-mL RBF, to a solution of the crude product (0.95 g, based on 3.75 mmol, 1 eq) and KSCN (4.37 g, 45.0 mmol, 12 eq) in 20 mL AcOH was added bromine (0.19 mL, 3.75 mmol, 1 eq) dropwise. This mixture was stirred at r.t. for 2 h and was then diluted with H₂O (60 mL), neutralised with aqueous NaOH (30% wt) and extracted with EtOAc (3×80 mL). The combined organic layers were dried over MgSO₄, filtered and evaporated under reduced pressure. The crude material was purified by column chromatography (30 g silica, EtOAc) yielding the title compound as a yellow oil (690 mg, 61%).

¹H NMR (CDCl₃, 400 MHz) δ_{H} 7.16 (d, J 1.6 Hz, 1H), 7.11 (dd, J 1.6 Hz, 8.8 Hz, 1H), 7.01 (br. s, 1H), 6.94 (d, J 8.8 Hz, 1H), 4.10 (dd, J 5.8 Hz, 5.8 Hz, 2H), 3.71 (dd, J 5.8 Hz, 5.8 Hz, 2H).

¹³C NMR (CDCl₃, 100 MHz) δ_{C} 161.0 (1C), 143.7 (q, J 2.0 Hz, 1C), 139.2 (1C), 123.7 (1C), 120.3 (q, J 256.9 Hz, 1C), 119.6 (1CH), 115.2 (1CH), 109.5 (1CH), 48.4 (1CH₂), 42.5 (1CH₂).

¹⁹F NMR (CDCl₃, 376 MHz) δᵣ-58.4.

IR υₘₐₓ 3044, 2929, 2110, 1610, 1584, 1485, 1256.

HRMS m/z (ESI⁺) calculated for C₁₀H₈F₃NOS[M+H]⁺: 304.05, found 304.05.

### Methyl 2-allyl-1-((1-(2-(2-imino-6-(trifluoromethoxy)benzo[d]thiazol-3(2H)-yl)ethyl)-1H-1,2,3-triazol-4-yl)methyl)-3-oxoindoline-2-carboxylate 40

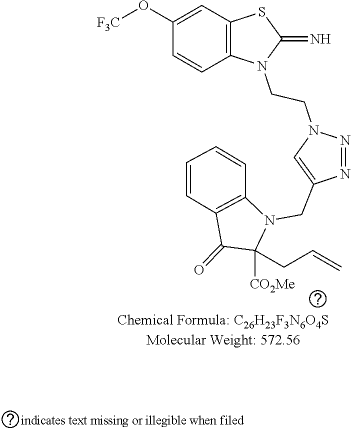 [SMILES: C=CCC1(C(=O)OC)C(=O)c2ccccc2N1Cc1cn(CCn2c(=N)sc3cc(OC(F)(F)F)ccc32)nn1]

To a solution of 3-N-(azidoethyl)riluzole (45 mg, 0.15 mmol, 1.0 eq) and methyl 2-allyl-3-oxo-1-(prop-2-yn-1-yl)indoline-2-carboxylate (60 mg, 0.22 mmol, 1.5 eq) in a 1:1 mixture of THF (3 mL) and H₂O (3 mL) stirring at r.t. was added dropwise an aqueous solution of CuSO₄.5H₂O (37 mg, 0.15 mmol, 1.0 eq) and a freshly prepared aqueous solution (0.2 mL H₂O) of sodium ascorbate (59 mg, 0.30 mmol, 2.0 eq). After 1 h (total consumption of azide by TLC), aqueous NH₄OH (28%, 15 mL) was added to the reaction mixture which was then extracted with DCM (3×50 mL). The combined organic layers were dried over MgSO₄, filtered and evaporated under reduced pressure. The crude material was purified by column chromatography (15 g silica, Petrol:EtOAc 1:1, to EtOAc to EtOAc:MeOH 9:1, v/v) yielding the title compound as a bright yellow solid (67 mg, 78%). methyl 2-allyl-3-oxo-1-(prop-2-yn-1-yl)indoline-2-carboxylate was also isolated (12 mg, 20%, 60% of max theoretical yield).

¹H NMR (CDCl₃, 400 MHz) δ_{H} 7.57 (d, J 7.6 Hz, 1H), 7.31-7.35 (m, 2H), 7.10 (d, J 1.6 Hz, 1H), 7.02 (br. s, 1H), 6.89 (d, J 8.8 Hz, 1H), 6.78 (app t, J 7.4 Hz, 1H), 6.53 (d, J 8.3 Hz, 1H), 6.44 (d, J 8.8 Hz, 1H), 5.24-5.35 (m, 1H), 5.01 (dd, J 1.4 Hz, 17.0 Hz, 1H), 4.90 (d, J 10.1 Hz, 1H), 4.72 (t, J 5.8 Hz, 2H), 4.58 (d, J 16.7 Hz, 1H), 4.46 (d, J 16.7 Hz, 1H), 4.40 (t, J 5.8 Hz, 2H), 3.65 (s, 3H), 3.02 (dd, J 6.7 Hz, 14.8 Hz, 1H) 2.85 (dd, J 7.5 Hz, 14.8 Hz, 1H).

¹³C NMR (CDCl₃, 100 MHz) δ_{C} 194.7 (1C), 167.7 (1C), 160.71 (1C), 160.67 (1C), 144.6 (1C), 143.7 (q, J 2.0 Hz, 1C), 138.5 (1C), 137.8 (1CH), 130.5 (1CH), 125.1 (1CH), 123.6 (1CH), 123.4 (1C), 120.4 (q, J 257.5 Hz, 1C), 120.0 (1CH₂), 119.7 (1CH), 119.5 (1C), 118.6 (1CH), 115.1 (1CH), 109.3 (1CH), 108.8 (1CH), 77.4 (1C), 53.1 (1CH₃), 47.0 (1CH₂), 43.6 (1CH₂), 40.4 (1CH₂), 37.2 (1CH₂).

¹⁹F NMR (CDCl₃, 376 MHz) δᵣ-58.4.

IR υₘₐₓ (thin film, cm⁻¹): 2920, 2851, 1739, 1703, 1613, 1583, 1484.

HRMS m/z (ESI⁺) calculated for C₂₆H₂₃F₃N₆O₄S [M+H]⁺: 573.1526, found 573.1524.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following Claims.

1. A compound according to formula (3): 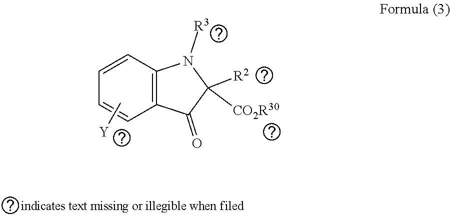
wherein
wherein R3 represents a 2-alkenyl or a 1-allenyl group;
each Y group independently represents H, halogen, an alkoxy group, an optionally substituted alkyl group, an optionally substituted (CH₂)K-carbocyclyl group, or an optionally substituted (CH₂)K-aryl group, where K represents an integer from 0 to 6;
R8 represents an optionally substituted hydrocarbyl group;
R20 represents an alkyl group.

2. A compound of the formula (I): 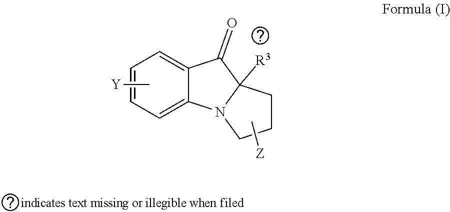
wherein
R3 represents a 2-alkenyl or a 1-allenyl group;
each Y group independently represents H, halogen, an optionally substituted hydrocarbyl group, an alkoxy group, an optionally substituted (CH₂)K-carbocyclyl group, or an optionally substituted (CH₂)K-aryl group, where K represents an integer from 0 to 6;
each Z group independently represents H, halogen, an optionally substituted hydrocarbyl group, or an alkoxy group.

3. The compound as claimed in claim 1, wherein R3 represents a 2-alkenyl group including 3 to 7 carbon atoms, or a 1-allenyl group including 3 to 5 carbon atoms.

4. The compound as claimed in claim 1, wherein R3 represents an unbranched 2-alkenyl group including 3 to 10 carbon atoms; preferably 3 or 4 carbon atoms.

5. The compound according to claim 1 having the structure of Formula (3a): 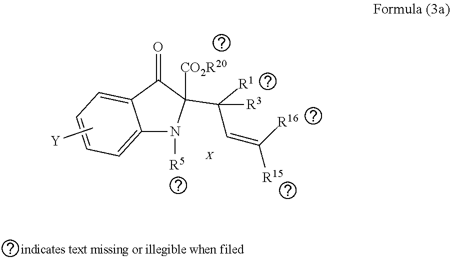
wherein
R1, R2, R15, R16 and X independently represent H, or an optionally substituted hydrocarbyl group, where two or more of the R1, R2, R15 and R16 groups may combine to form a carbocyclyl group.

6. The compound as claimed in claim 1 having the structure of Formula (3b): 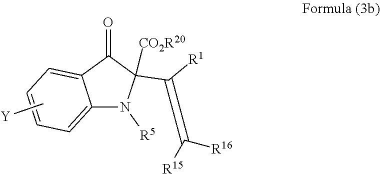
wherein
R1, R15 and R16 independently represent H, or an optionally substituted hydrocarbyl group, where two or more of the R1, R15 and R16 groups may combine to form a carbocyclyl group.

7. The compound according to claim 2 having the structure of Formula (1a): 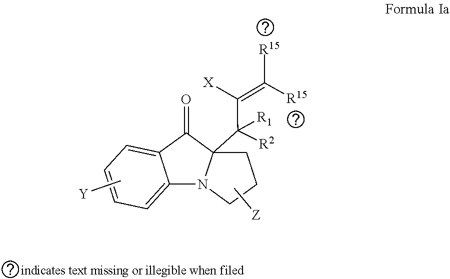
wherein
R1, R2, R15, R16 and X independently represent H, an optionally substituted hydrocarbyl group (such as an alkyl group), where two or more of the R1, R2, R15 and R16 groups may combine to form a carbocyclyl group.

8. The compound according to claim 2 of the formula (Ib): 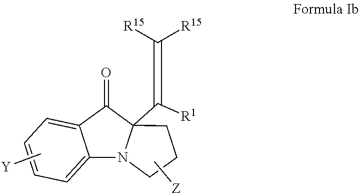
wherein
R1, R15 and R16 independently represent H, an optionally substituted hydrocarbyl group (such as an alkyl group), where two or more of the R1, R15 and R16 groups may combine to form a carbocyclyl group.

9. The compound as claimed in claim 1 wherein each Y group independently represents H, a straight chain alkyl group including 1 to 6 carbon atoms, an alkoxy group having the structure —O—C_{1 to 6} alkyl group, or a substituted or unsubstituted C5 to 7 aryl group, generally having the structure —(CH₂)_{K}-aryl group, where K is an integer from 0 to 6.

10. The compound as claimed in claim 1 wherein each Y group independently represents H, methyl, methoxy or a phenyl group.

11. The compound according to claim 1, wherein R8 represents an alkyl, alkylene, alkynyl, carbocycle, or aryl group.

12. The compound according to claim 1, wherein R8 represents a straight chain alkyl group having 1 to 6 carbon atoms, an aryl ring moiety having 5, 6 or 7 carbon ring atoms, in particular an optionally substituted (CH₂)L aryl group or an optionally substituted (CH₂)L heteroaryl group where L represents an integer from 1 to 10; a straight chain alkylene group having a carbon backbone of 3 to 6 carbon atoms, a straight chain alkynyl group having a carbon backbone of 3 to 6 carbon atoms, or a heteroaryl group.

13. The compound according to claim 2, wherein Z represents a hydrocarbyl group substituted with one or more oxygen, halogen or nitrogen group, generally a Cl to 6 hydrocarbyl group substituted with one or more oxygen, fluorine, iodine, or nitrogen group.

14. The compound as claimed in claim 1 wherein the compound is substantially uncharged.

15. The compound as claimed in claim 1 having a Stokes shift of more than 110 nm.

16. The compound as claimed in claim 1 having a number average molecular weight of 100 to 650 g/mol.

17. A method of synthesizing a compound as claimed in claim 1 according to reaction scheme A: 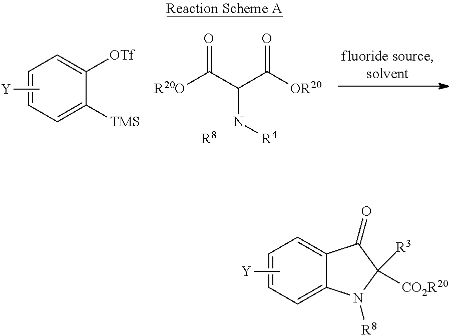
Wherein R4 represents a 2-alkenyl group, or a 2-alkynyl group,
OTf represents trifluoromethane sulfonate; and
TMS represents trimethylsilyl.

18. A method of synthesizing a compound as claimed in claim 2 according to reaction scheme (1): 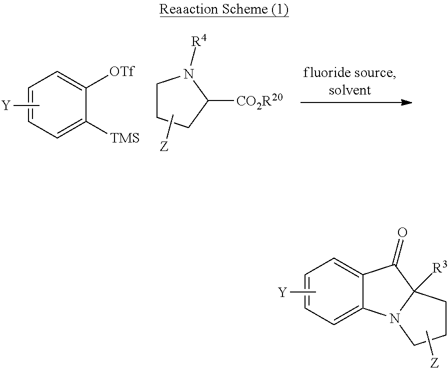
Wherein R4 represents a 2-alkenyl group, or a 2-alkynyl group;
OTf represents trifluoromethane sulfonate; and
TMS represents trimethylsilyl.

19. A fluorescent label, comprising the compound as claimed in claim 1.

20. A method of ex-vivo or in-vitro cell imaging, comprising the step of providing the fluorescent label as claimed in claim 19.

21. A method of tagging a compound comprising an amine group comprising contacting the compound comprising an amine group with a compound as claimed in claim 1 and monitoring fluorescence emitted.

22. A method of measuring the amount of binding between a molecular target drug and a biological target wherein the molecular target drug comprises an amine group, including reacting the molecular target drug with the compound as claimed in claim 1, administering the resultant product to a sample comprising the biological target and monitoring fluorescence emitted from the sample.
